(19) United States
(12) Patent Application Publication
(10) Pub. No.: US 2025/0073505 A1
(43) Pub. Date: Mar. 6, 2025

(54) ACOUSTIC DEVICES WITH ADJUSTABLE PENETRATION DEPTH AND OPTICAL OBSERVATION

(71) Applicant: TOOSONIX A/S, Hoersholm (DK)

(72) Inventors: Tomasz Zawada, Hoersholm (DK); Torsten Bove, Hoersholm (DK)

(21) Appl. No.: 18/952,936

(22) Filed: Nov. 19, 2024

Related U.S. Application Data
(63) Continuation of application No. PCT/EP2023/063766, filed on May 23, 2023.
(60) Provisional application No. 63/344,983, filed on May 23, 2022.

(51) Int. Cl. A61N 7/00

(52) U.S. Cl. CPC A61N 7/00; A61N 2007/0052

(57) ABSTRACT

Acoustic generating systems are provided. The acoustic generating systems include a handpiece that provides for adjustment of the penetration depth. In preferred embodiments, the adjustment of the penetration depth is provided by adjustment between a fixed part of the handpiece and an adjustable part of the handpiece. In even more preferred embodiments, adjustment of the penetration depth is provided through a plurality of interchangeable adjustable parts each configured to provide for a different penetration depth when coupled to the fixed part. Preferred embodiments further include an optical system that provides imaging of the treatment surface and is capable of adjusting to the change in penetration depth to continue to provide imaging of the treatment surface. In some embodiments, the interface between the fixed part and adjustable part of the handpiece passes through a chamber that holds a liquid acoustic coupling medium.

### CROSS-REFERENCE TO RELATED APPLICATIONS

This continuation application claims priority to International Patent Application PCT/EP2023/063766, filed on May 23, 2023, which has published as WO 2023/227588, which claims priority to U.S. Provisional Patent Application 63/344,983, filed May 23, 2022, the entire contents of which are fully incorporated herein with these references.

### FIELD

This patent document relates to acoustic devices and their use in methods and systems for treatment of the human body, in particular treatment of human skin.

### BACKGROUND

Focused ultrasound can be used for non-invasive treatment of internal organs in the human body. The focused acoustic energy is transferred through the skin surface to the target tissue, where it is absorbed causing thermal as well as mechanical interaction with the targeted organ/tissue.

In recent years high intensity focused ultrasound (HIFU) has become a therapeutic method for treatment of numerous diseases. In most cases the treatment zone is located deep in the human body. Removal of tumors in brain, prostate, thyroid glands, or uterine fibroids have been the major areas of interest. Accordingly, HIFU is usually accompanied by an imaging method enabling observation as well as targeting of the pathological tissue to be treated. Magnetic Resonance Imaging (MRI) and ultrasonic imaging are by far the most popular modalities used in experimental treatment, as well as in commercial systems. The cost and complexity of HIFU systems, and therefore their ubiquity, are greatly impacted by the need for complex imaging systems, especially in the case of MRI.

Recently new methods for treatment of human skin using HIFU as the therapeutic modality combined with optical imaging has been proposed. Human skin is usually treated using cryotherapy or laser-therapy. Cryotherapy is by far the cheapest and the most ubiquitous. However, cryotherapy does not allow for precise control of the depth of treatment. Due to surface temperature distribution, the center of the treated area is usually overtreated while the peripheries are undertreated, which can greatly increase the probability of low efficacy and post-treatment scarring. Similarly, lasers in general require good absorption of light, which is not the case in many diseases. Moreover, the depth of treatment strongly depends on the local emissivity that can lead to local overexposures again leading to low efficacy and increased probability of scarring. Probability of infection is also increased as the laser therapy is exposing basically an open wound to the outside environment.

The new methods of HIFU treatment of human skin allow for more targeted treatments where the energy is deposited at the exact level within the dermis or epidermis, creating flat and more controllable treatment areas. In addition, the surface of the skin is left intact as the point of treatment is located beneath it due to the focused nature of the ultrasonic wave. This aids the healing process and reduces the probability of infections. A wide range of skin conditions can be addressed by the proposed treatment beginning with cancer conditions such as Basal Cell Carcinoma, Squamous Cell Carcinoma or Kaposi's Sarcoma, pre-cancer condition such as Actinic Keratosis, and others like Seborrheic Keratosis, Sebaceous Hyperplasia, ending with milder conditions such as vascular malformations, viral warts, acne, and many more within the general category of epidermal and cutaneous neoplasms.

FIG. 1 illustrates the basic physiology of human skin 51. Human skin 51 is composed of an outer epidermis-layer 40, which is separated by the basement membrane 52 towards the deeper dermis 41 layer and even deeper subcutaneous layer 53.

Throughout the dermis 41 and subcutaneous 53 layers, lymphatic channels and vasculature 54 provide transport of nutrients, cells and various pathogens to and from the skin 51. The transport of dead cells away from the dermis-layer 41 is facilitated by macrophages, which are a type of white blood cell. In a process called phagocytosis, the macrophages engulf and digest cellular debris, foreign substances, microbes, cancer cells, and anything else that does not have the types of proteins specific of healthy body cells on its surface.

The basement membrane 52 controls the traffic of the liquid, molecules and cells between the dermis 41 and epidermis 40, but also serves as a reservoir for their controlled release during physiological remodelling or repair processes. As the epidermis 40 contains no blood vessels, transport through the basement membrane 52 to the epidermis 40 is limited by the diffusion-processes, which thereby also effectively limits transport of larger particles, cells, or chemicals, such as pigments, through this barrier. The thickness of the above-mentioned three outer layers of the skin is typically in the range of 0.1 mm to 5.0 mm depending on the location on the body.

HIFU devices for cosmetic treatment of skin (deep dermis and below) operating at frequencies up to 4 MHz-12 MHz with built-in ultrasonic imaging are known in the prior art. However, ultrasound imaging guidance is less relevant as features located on the skin surface give better guidance for treatment. It is argued that a very well defined and small focal zone created by the properly designed transducer is the prerequisite for appropriate treatment of the skin.

FIG. 3 outlines an example of an ultrasonic device 57, where a focused acoustic source 31 is coupled to a housing 1 and produces focused ultrasonic field with focal point 21 at an acoustic focal distance 33. A coupling medium 58 is placed between the focused acoustic source 31 and the surface of the skin 55 inside the housing 1.

Each skin disease can be attributed its origin in the skin layers or functional elements. For example, basal cell carcinoma originates in the basement membrane, while squamous cell carcinoma in the squamous layer of the epidermis 40, on the other hand acne originates in sebaceous glands located typically within dermis. Therefore, it is advantageous to perform a skin treatment selectively affecting only the pathological tissue, saving other layers or fragments of the healthy skin. This leads to faster healing, lower probability of infections and lower likelihood of scar formation. The selectivity of skin treatment can be provided by selection of the point-of-treatment (POT) 56.

In various embodiments, it is advantageous to use focused ultrasound for selective treatment of skin where acoustic focal point 21 coincides with pre-selected POT 56. The distance between the surface of the skin 55 and the focal point acoustic focal point 21 is called penetration depth 17.

In various embodiments it is advantageous to fabricate a transducer with adjustable penetration depth as that would enable use of the same device (handpiece) to target tissue at different penetration depths, thus broadening the application range of a single device.

This problem has been addressed in the prior art by several mechanisms such as varying focal length 33 of the focused transducer. This can be achieved both mechanically and electronically. The mechanical techniques include defocusing or focusing using acoustic lenses, reflectors combined with adjustment of temporal field. Alternatively, the electronic focusing can be performed using an annular array device, where effective focal distance is predetermined by a time delay of the driving signal reaching each individual annulus. The aforementioned techniques, however, bear significant costs, lead to the increased weight and size as they require either complex mechanical setups or costly multichannel driving electronics. Moreover, the varying focal distance affects the shape and size of the focal zone created by the focused transducer, as the longer the focal distance the larger the F-number resulting in larger volumes of the focal zones. Larger focal zones compromise the treatment resolution because the treatment is performed deeper into the skin.

What is needed is an ultrasonic device that is designed in a more cost effective way to allow the depth of the POT to be adjusted without compromise to the size and shape of the focal zone. It would be further beneficial to include an imaging system with the device that could similarly be adjusted to track the location of the POT or corresponding point on the surface of the skin.

### SUMMARY OF THE EMBODIMENTS

Objects of the present patent document are to provide an improved apparatus and methods for the treatment of tissue, in particular human skin. The embodiments herein use unique and novel acoustic generating systems that provides for adjustment of the penetration depth. In preferred embodiments, the adjustment of the penetration depth is provided by adjustment between a fixed part of the handpiece and an adjustable part of the handpiece. In even more preferred embodiments, adjustment of the penetration depth is provided through a plurality of interchangeable adjustable parts each configured to provide for a different penetration depth when coupled to the fixed part. Preferred embodiments further include an optical system that provides imaging of the treatment surface and is capable of adjusting to the change in penetration depth to continue to provide imaging of the treatment surface. In some embodiments, the interface between the fixed part and adjustable part of the handpiece passes through a chamber that holds a liquid acoustic coupling medium.

In some embodiments, an acoustic generating system is provided. The acoustic generating system comprises a fixed part of a handpiece wherein the fixed part includes a focused acoustic generating source with a fixed acoustic focal length. The acoustic generating system comprises a plurality of adjustable parts for the handpiece wherein each adjustable part in the plurality of adjustable parts is designed to be removably and interchangeably coupled to the fixed part of the handpiece, one at a time. In preferred embodiments, a penetration depth of the handpiece is adjusted by coupling different adjustable parts in the plurality of adjustable parts with the fixed part. This may be achieved by providing adjustable parts with different total lengths along their longitudinal axis.

In preferred embodiments, the fixed part of the handpiece further comprises an optical system that provides imaging of a treatment surface through any adjustable part in the plurality of adjustable parts that is coupled to the fixed part.

In embodiments with an optical system, the optical system may comprise an objective and an imaging plane and wherein a distance along a longitudinal axis between the objective and the imaging plane is adjustable. In some embodiments, the distance along the longitudinal axis between the objective and the imaging plane is adjusted by mechanically moving the objective along the longitudinal axis towards or away from the imaging plane.

In some embodiments, an optical principal axis of the optical system is coaxial with a main acoustic axis of the focused acoustic generating source. In other embodiments, the optical system has an optical main axis that includes a portion that is not coaxial with an acoustic main axis.

In some embodiments with an optical system, the optical system comprises an imaging plane and at least one optical component with an adjustable optical focal length along a longitudinal axis. In some embodiments with an adjustable optical focal length, the adjustment is achieved by a liquid lens. In other embodiments, the adjustable optical focal length is achieved by one of a group consisting of electrowetting, use of shape-changing polymers and acousto-optical tuning.

In some embodiments, he penetration depth is adjustable between at least 0 mm and 5 mm and an optical depth of field of the optical system is equal to or greater than 5 mm. Other ranges for the adjustment of the penetration depth and the depth of field are possible.

In some embodiments, an interface between the fixed part of the handpiece and any adjustable part in the plurality of adjustable parts passes through a chamber that is designed to contain a liquid acoustic coupling medium.

In some configurations, an acoustic generating system is provided that comprises a fixed part of a handpiece and a plurality of adjustable parts for the handpiece wherein each adjustable part in the plurality of adjustable parts is designed to be removably and interchangeably coupled to the fixed part of the handpiece, one at a time. The interface between the fixed part of the handpiece and any adjustable part in the plurality of adjustable parts passes through a chamber that is designed to contain a liquid acoustic coupling medium.

### BRIEF DESCRIPTION OF THE DRAWINGS

FIG. 1 illustrates a cross-section of a human skin and a point of treatment (POT) located underneath the skin surface.

FIG. 2 illustrates the acoustic field produced by a focused acoustic source together with basic parameters of the focal zone.

FIG. 3 illustrates a schematic view of an acoustic device comprising a housing with a focused acoustic source coupled through a coupling medium to a tissue (e.g. skin) with the acoustic focal point located beneath the surface of the tissue.

FIG. 4 illustrates an acoustic device having a housing comprising a fixed part and an adjustable part coupled to a tissue (e.g. skin) depositing acoustic energy into selected regions of the internal structures (e.g. vein) at fixed acoustic focal length of the focused acoustic source.

FIG. 5 illustrates the definition of an optical focal length of a focusing optical lens.

FIG. 6 illustrates an image formation of an object by an optical thick lens.

FIG. 7 illustrates an image formation of an object by a complex optical system, comprising optical lenses (e.g. an optical objective).

FIG. 8 illustrates the definition of an optical depth of field and an optical depth of focus of an optical imaging system comprising optical lenses (e.g. an objective).

FIG. 9 illustrates major parameters of an image sensor.

FIG. 10 illustrates an example of a lens system suitable for imaging of the surface of a tissue (e.g. skin) through transparent acoustic path provided by a focused acoustic source (dimensions given in mm).

FIG. 11 illustrates the change of field of view due to refraction of light rays going through three layers of transparent materials of different refractive index.

FIG. 12 illustrates the objective adjustment due to presence of an acoustically and optically transparent coupling medium in the optical path.

FIG. 13A illustrates an example of an embodiment comprising a focused acoustic source with a transparent optical path and an objective forming an image of a surface of the tissue to be treated (e.g. skin).

FIG. 13B illustrates an example of an embodiment comprising a focused acoustic source with a transparent optical path and an objective forming an image of a surface of the tissue to be treated (e.g. skin). An adjustment of the penetration depth (shallower than in FIG. 13A) requires change of the optical objective position, so it is moved towards the image plane, provided an objective of a fixed optical focal length is used.

FIG. 14A illustrates an example of an embodiment comprising a focused acoustic source with a transparent optical path and an objective forming an image of a surface of the tissue to be treated (e.g. skin).

FIG. 14B illustrates an example of an embodiment comprising a focused acoustic source with a transparent optical path and an objective forming an image of a surface of the tissue to be treated (e.g. skin). An adjustment of the penetration depth (shallower than in FIG. 14A) requires change of the focal length of the optical objective, provided that the objective is at fixed position in relation to the image plane.

FIG. 15 illustrates an example of an embodiment comprising a focused acoustic source with a transparent optical path and an objective forming an image of a surface of the tissue to be treated (e.g. skin). A part of the optical main axis of the optical objective direction is not coaxial with the acoustic main axis.

FIG. 16 illustrates an example of an embodiment comprising a focused acoustic source with a transparent optical path and an objective forming an image of a surface of the tissue to be treated (e.g. skin).

FIG. 17 illustrates the important elements of a focused acoustic source coupled to the fixed part of the housing.

FIG. 18 illustrates an example of a focused acoustic source coupled to the fixed part of the housing coupled with an adjustable part of the housing. Direction of the coupling is shown by the arrow.

FIG. 19 illustrates an example of a focused acoustic source coupled to the fixed part of the housing coupled with an adjustable part of the housing. The coupling between the fixed and adjustable part of the housing is made watertight using a seal.

FIG. 20 illustrates an example of an embodiment comprising a focused acoustic source with a transparent optical path coupled to a fixed part of the housing comparing a seal and a fixing groove and ridge. The adjustable part of the housing features a round opening, and the bubble trap. The adjustable part of the housing is shown before it is affixed onto the fixed part of the housing.

FIG. 21 illustrates an example of an embodiment comprising a focused acoustic source with a transparent optical path coupled to a fixed part of the housing comprising a seal and a fixing groove. The adjustable part of the housing features a round opening, and a bubble trap. The adjustable part of the housing is shown after it has been affixed onto the fixed part of the housing.

FIG. 22 illustrates an example of an embodiment comprising a focused acoustic source with a transparent optical path coupled to a fixed part of the housing, and a movable optical objective and an image sensor. The adjustable part of the housing features a round opening, and a bubble trap.

FIG. 23 illustrates an example of an embodiment comprising a focused acoustic source with a transparent optical path coupled to a fixed part of the housing, a movable optical objective in a threaded fixture and an image sensor. The adjustable part of the housing features a round opening, and a bubble trap. The adjustable part of the housing is coupled to the fixed part of the housing using threaded coupling enabling a continuous adjustment of the penetration depth.

FIG. 24 illustrates an example of an embodiment comprising a focused acoustic source with a transparent optical path coupled to a fixed part of the housing, a movable optical objective in a threaded fixture and an image sensor. The threaded objective is coupled magnetically to the external ring allowing for optical focus adjustment. The adjustable part of the housing features a round opening, and a bubble trap. The adjustable part of the housing is coupled to the fixed part of the housing using threaded coupling enabling a continuous adjustment of the penetration depth.

FIG. 25 is a table of different configurations of the focused acoustic source resulting in different parameters of the acoustic focal zone.

FIG. 26 is a table of different configuration of the image sensors and distances to the object and to the image resulting in different field of view, total optical track lengths and magnifications.

FIG. 27 is a table of different configurations of imaging media and optical objectives resulting in different combinations of an optical resolution, a numerical aperture, an optical depth of field and an optical F-number.

FIG. 28 depicts an example of a displayed real-time image of the surface of the skin with an augmented reality overlay showing an outline of the lesion to be treated as well as the treatment points.

FIG. 29 illustrates an example of a displayed real-time image of the surface of the skin with an augmented reality overlay showing an outline of the blood vessel created using data fusion techniques from ultrasonic imaging data.

FIG. 30 depicts a block diagram of a therapeutic system comprising a main unit and a handpiece connected through a wired connection.

FIG. 31 shows a block diagram of a therapeutic system comprising a main unit and a standalone handpiece connected through a wireless connection.

FIG. 32 depicts a block diagram of a handheld therapeutic system a standalone handpiece.

FIG. 33 shows the principle of continuous adjustment of the penetration depth.

FIG. 34 illustrates an example of a configuration with motorized adjustment of the penetration depth.

FIG. 35 illustrates an architecture of a handpiece.

### DETAILED DESCRIPTION OF THE PREFERRED EMBODIMENTS

Several embodiments disclosed herein combine focused acoustic energy and optical imaging. From a physics perspective, both acoustics and optics deal with the wave phenomena governed by diffraction, reflection and refraction, and as such, share a lot of terms used to describe individual elements and effects. Throughout this document elements, phenomena, parameters, etc. related to acoustics have an adjective ‘acoustic’. Similarly, elements, phenomena, parameters, etc. related to optics have an adjective ‘optical’, in order to avoid possible misinterpretation. Moreover, throughout this document, the term ‘acoustic’ is understood as related to mechanical waves of frequency from 1 Hz to 1 GHz, and ‘optical’ is understood as related to electromagnetic waves of wavelength from 10⁻¹⁰ m to 10⁻³ m.

### Acoustic Field of a Focused Acoustic Source

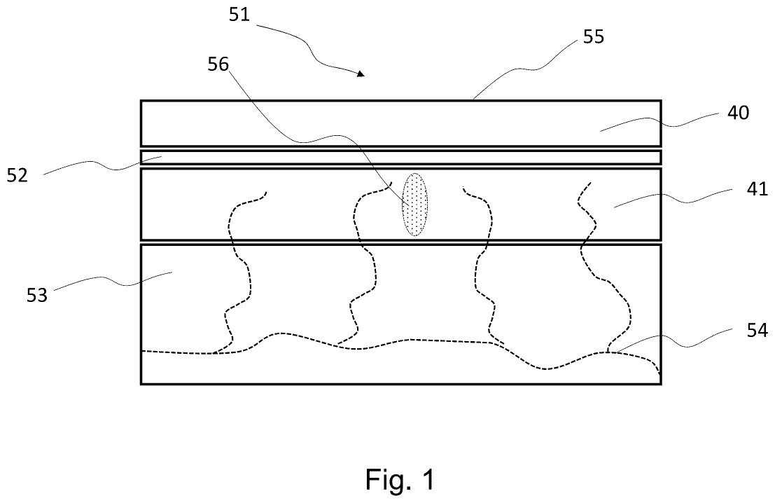

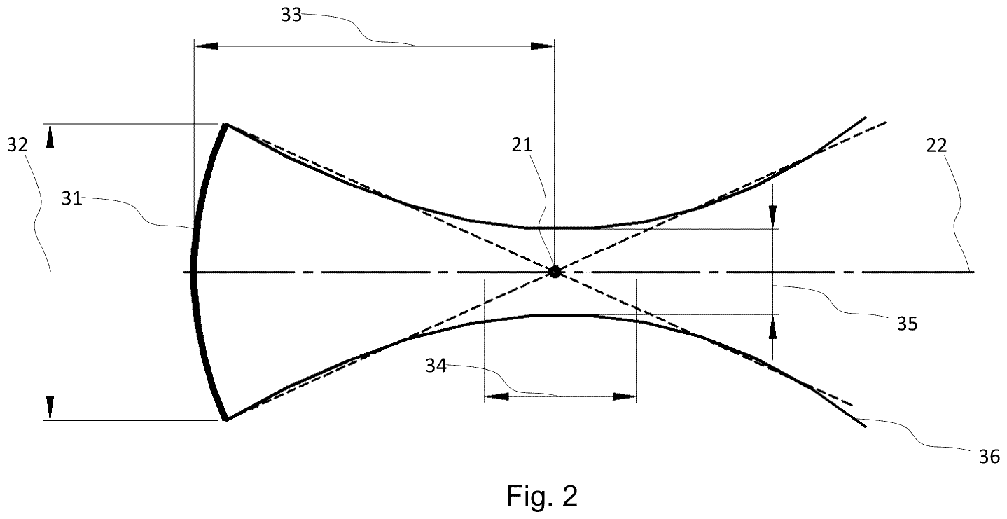

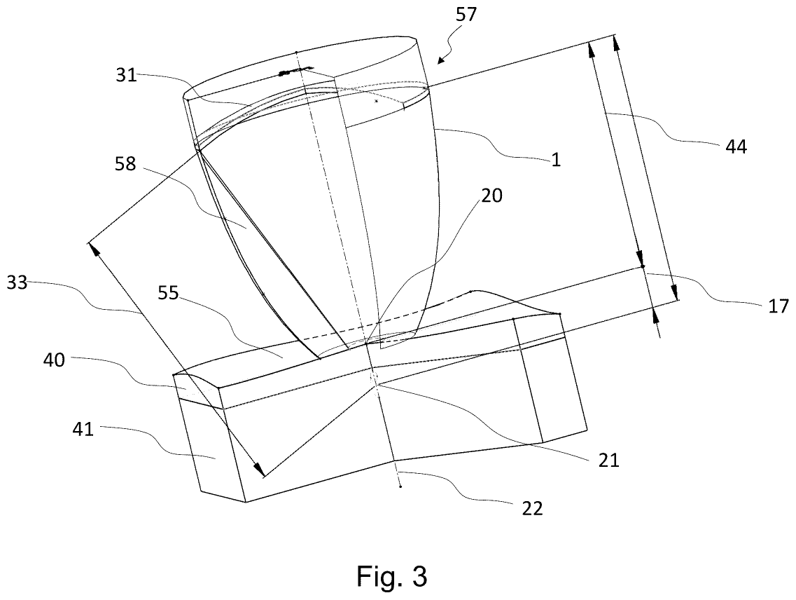

In several embodiments disclosed herein a focused acoustic source 31 is utilized to produce a focal point 21 of high acoustic intensity within tissue to be treated 51, as may be seen in FIGS. 1 and 3. In preferred configurations, the acoustic focal point 21 is located on the acoustic main axis 22 at the acoustic focal length 33. FIG. 2 illustrates the contour of a focused acoustic field 36 produced by a focused acoustic source 31. The resulting focused acoustic field 36 produces an acoustic focal zone which is a volume around the focal point 21 defined by a drop in pressure (decrease) of 6 dB compared to the maximum pressure at the acoustic focal point 21. The dimensions of the acoustic focal zone are the acoustic depth of focus 34 and acoustic focal diameter 35. As shown in equation 1 and 2, there is a relation between the acoustic depth of focus DOFₐ34 and the acoustic focal diameter FDₐ35 and the properties of the focused acoustic source 31 given by the operation frequency f₀, aperture α=D/2, and acoustic focal length (33) Fₐ, where D is the diameter of the acoustic focused source 32.

$$D O {F}_{a} = 2.39 \times {λ}_{0} {( \frac{{F}_{a}}{a} )}^{2} = 9.56 \times {λ}_{0} {( {F}_{\#} )}^{2} \qquad ( 1 )$$

$$F {D}_{a} = 0.70 \times {λ}_{0} ( \frac{{F}_{a}}{a} ) = 1.40 \times {λ}_{0} ( {F}_{\#}^{a} ) \qquad ( 2 )$$

Acoustic wavelength λ₀=cₐ/f₀, where cₐ is the speed of sound in the given medium. In some embodiments the medium is water and cₐ=1480 m/s. Acoustic F-number F_{#}^{a}=Fₐ/D.

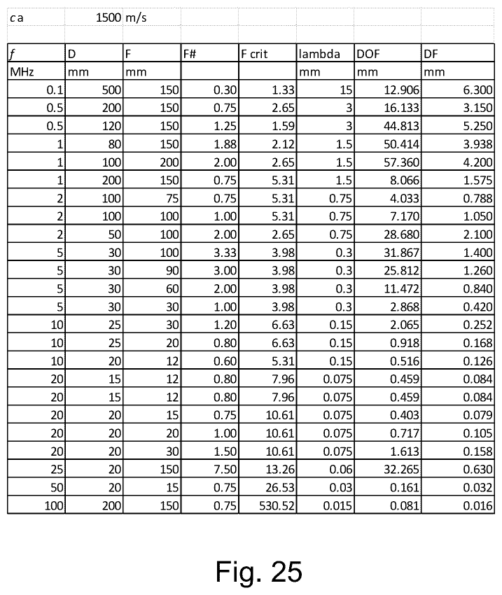

In preferable embodiments the size of the acoustic focal zone should be matched with the expected treatment protocol, in particular the size of the organ or tissue to be treated. In some embodiments a focused acoustic field is used to treat selected layers of human skin, known to be between 1-5 mm thick. In such cases, the acoustic depth of focus 34 can be achieved by appropriate selection of an operation frequency f₀. The operation frequency f₀ can range from 0.1 MHz to 1 GHz, or 1 MHz to 100 MHz, or a variety of other ranges depending on the embodiment. The acoustic F-number defines the strength of focusing. The lower the acoustic F-number the stronger the focusing. Some embodiments may have F_{#}^{a} in the range from 0.5 to 100. More preferable embodiments may exhibit F_{#}^{a} from 0.51 to 20, or 0.55 to 10. One skilled in the art appreciates that the strong focusing is given by a criterium 3:

$${F}_{\#}^{a} < \frac{a}{4 {πλ}_{0}} = {F}_{\#}^{c} \qquad ( 3 )$$

where F^{c}_{#} is the critical value of acoustic F-number. In some preferable embodiments, it is expected that strong focusing is produced, i.e. that F_{#}^{a}<F^{c}_{#}. FIG. 25 illustrates possible combinations of operation frequency and acoustic F-number that can be used in the various embodiments described herein. The values in FIG. 25 are based on propagating focused ultrasound in water and soft tissue, assuming the speed of sound cₐ=1500 m/s. One skilled in the art appreciates that the speed of sound in soft tissue is slightly higher than in water, however this difference does not impact the overall ranges of the parameters of the preferable embodiments. FIG. 25 outlines several preferable configurations of the focused acoustic source.

### Acoustic Device Utilizing Focused Acoustic Source

FIG. 3 illustrates a schematic view of an acoustic device comprising a housing with a focused acoustic source 31 coupled through a coupling medium to a tissue (e.g. skin) with the acoustic focal point located beneath the surface of the tissue. The acoustic device comprises a focused acoustic source 31 coupled to a housing 1. In preferable configurations, the focal point 21 produced by the focused acoustic source 31 along the main acoustic axis 22 is located below the reference plane 20. Therefore, it is located within the organ or tissue to be treated when the reference surface 20 is coupled to the surface of the tissue to be treated 55. In some embodiments, the tissue or organ to be treated is a human skin including epidermis 40 and dermis 41.

In some embodiments, the acoustic focal point 21 is located below the reference surface 20. One skilled in the art appreciates that this can be achieved by a configuration where the acoustic focal length 33 is greater than the effective distance 44 between the apex of the focused acoustic source and the reference plane 20. The difference between the focal length and the distance 44 is equal to the penetration depth 17.

In preferred embodiments, the space between the focused acoustic source and the reference plane 20 within the housing 1 is filled with an acoustically transparent (i.e. having low acoustic attenuation) substance, i.e. acoustic coupling medium 58. In some embodiments this can be a fluid, a liquid, or a gas. In another embodiment, the acoustic coupling is water.

Acoustic Device with a Focused Acoustic Source and an Adjustable Penetration Depth

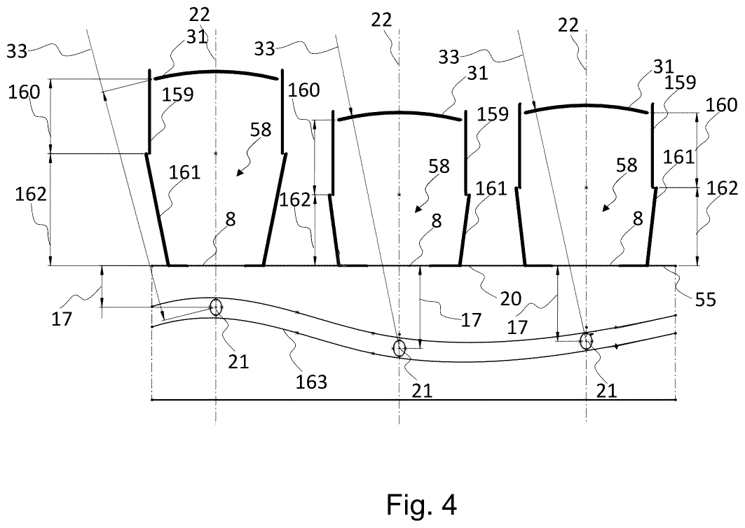

FIG. 4 illustrates a treatment of an internal structure 163 located at different depths below the surface 55 of the treated tissue (e.g. skin) using a device with adjustable penetration depth 17 in three different configurations. In the embodiment shown in FIG. 4, the housing 1 comprises more than one component that are adjustable along the longitudinal axis 22 with respect to one another. However, in other embodiments, the housing 1 may be comprised of more than two components that are adjustable along the longitudinal axis 22 with respect to one another. In the embodiment shown in FIG. 4, the housing 1 comprises two components: one coupled to the focused acoustic source 31, called the fixed part of the housing 159 and an adjustable part of the housing 161.

In embodiments like the one shown in FIG. 4, the distance between the apex of the focused acoustic source 31 and the reference plane 20 is equal to the length of the fixed part 160 and the length of the adjustable part 162 along the longitudinal axis 22. The sum of the lengths of the fixed 160 and adjustable 162 part of the housing together with penetration depth 17 equals to the acoustic focal length 33.

In some embodiments, the penetration depth is made adjustable by providing a plurality of replaceable adjustable parts 161 for the housing 1. In such embodiments, each adjustable part 161 in the plurality of replaceable adjustable parts has a different length 162 as measured along the longitudinal axis 22. In some embodiments, the adjustable length 162 of the different adjustable parts 161 in the plurality of adjustable parts is given in steps of 0.1 mm, 0.2 mm, 0.3 mm, 0.4 mm, 1 mm, 2 mm, 3 mm or any distance between 0.01 mm and 1000 mm. In some embodiments it is advantageous to match the adjustment step with the acoustic depth of focus of the focused acoustic source 31, given by eq. (1). In such case the adjustment step is equal to the fraction of the acoustic depth of focus 34. In some other embodiments the adjustment step is a multiplier of the acoustic depth of focus 34 from the range from 0.001 to 1000.

In some other embodiments, the housing 1 comprises more than two parts, more than three parts, or more than four parts. The parts can be moved along the acoustic axis 22 defining the length of the adjustable part 162. In addition to the movement along the main acoustic axis 22 the parts can rotate or move in any other direction to provide continuous adjustment of the length of the adjustable part 162 in the range from 0 mm to 1000 mm. In other embodiment the adjustment range is 0 mm to 100 mm or 0.01 mm to 20 mm. In the preferable embodiment adjustment range is from 1 mm to 10 mm.

In some embodiments, there may be a course adjustment mechanism and a fine adjustment mechanism. As just one non-limiting example, a three piece housing could have two adjustable parts 162. One of the adjustable parts could have a thread that allows for course adjustment with respect to the fixed body part, say 1 mm adjustments, and the second adjustable part may provide fine adjustments with respect to the fixed portion of the housing, say 0.1 mm adjustments.

### Optical Imaging Using Optical Devices

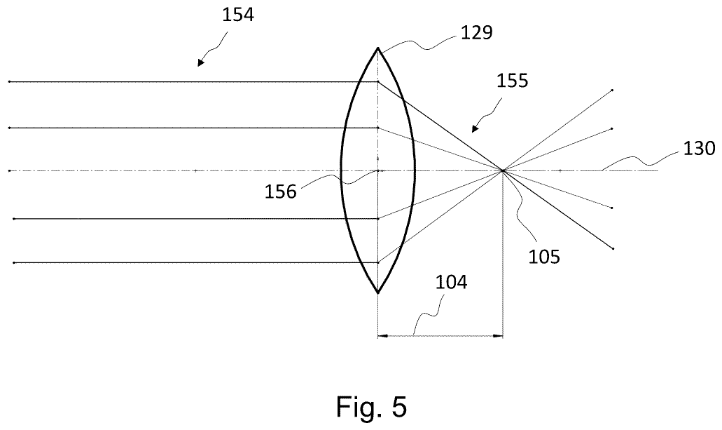

Preferred embodiments comprise optical devices providing continuous and real-time observation capabilities of the surface of the tissue to be treated (e.g. skin) 55. In a simple embodiment, a basic optical device is an optical lens 129. FIG. 5 shows basic properties of a double-convex optical lens characterized by an optical focal distance 104, which is the distance between the principal point 156 located on the optical principal axis 130, and the optical focal point 105. By definition, the optical focal point 105 is where the parallel incident light rays 154 meet after passing through the lens as a focused light beam 155.

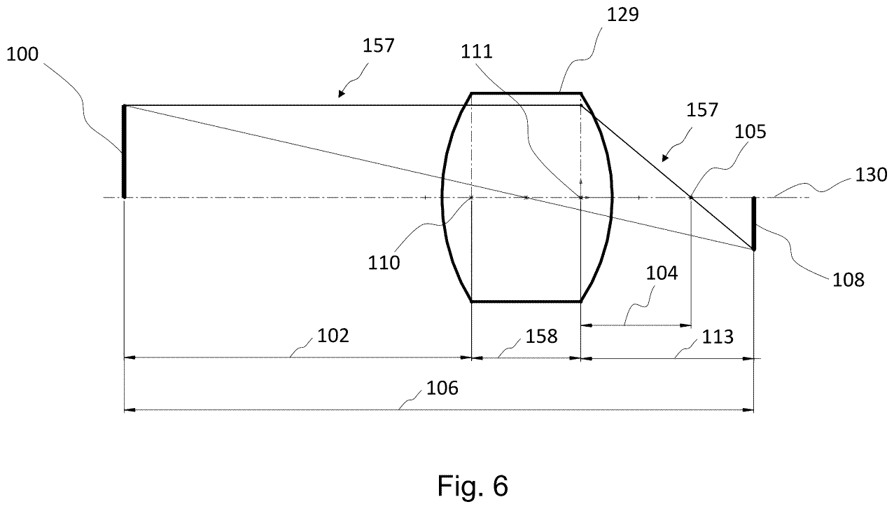

Most embodiments disclosed herein take advantage of a more complex optical system comprising a ‘thick’ optical lens setup, as shown in FIG. 6. A thick optical lens is a lens where the size of the lens zₗ 158 is defined by the distance between the primary principle point 110 and the secondary principal point 111. FIG. 6 depicts an optical double-convex lens 129 forming an image 108 at a distance zᵢ (113) from the secondary principle point 111 of an object 100 at a distance zₒ (102). The distance between the object 100 and the image 108 is called the total optical track length zₜ (106) and is given by the equation 4:

$${z}_{o} + {z}_{l} + {z}_{i} = {z}_{t} \qquad ( 4 )$$

One skilled in the art appreciates that a real image is not always formed by a system like the one shown in FIG. 6. However, if the right conditions are met for a given lens characterized by an optical focal length Fₒ(104) the following formula (5) holds:

$${F}_{o}^{2} = ( {z}_{i} - {F}_{o} ) \times ( {z}_{o} - {F}_{o} ) \qquad ( 5 )$$

The formula above links three major parameters of an optical system, providing two degrees of freedom.

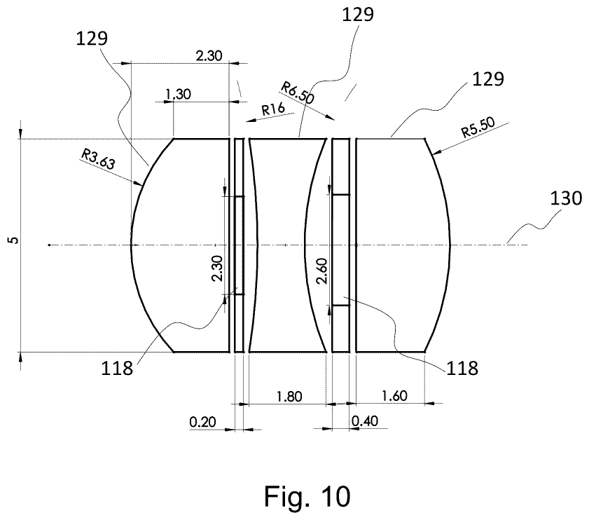

Several embodiments disclosed herein comprise a complex imaging system including a compound lens. A compound lens is a collection of simple lenses of different shapes and made of materials of different refractive indices, arranged in series along a common axis. FIG. 10 illustrates one example of a preferable configuration of a compound lens.

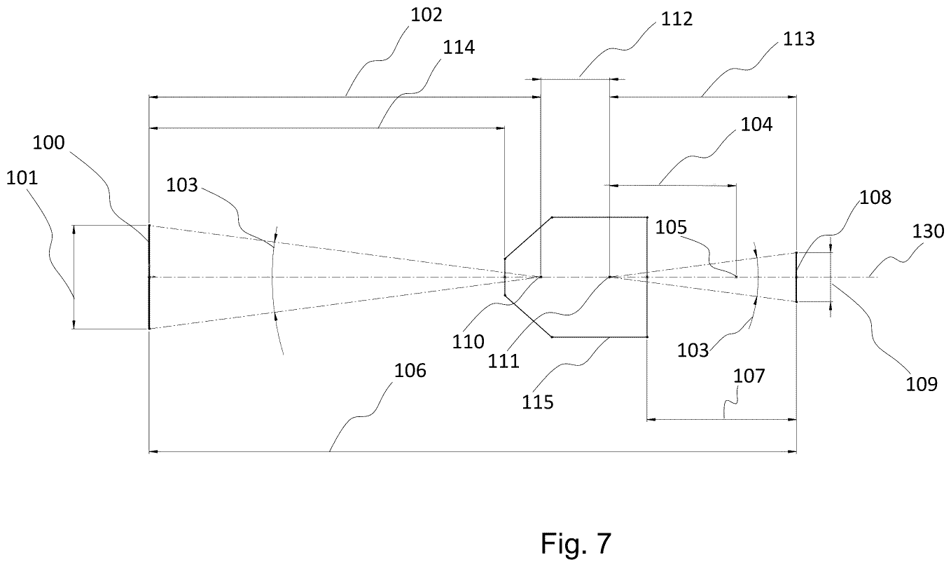

FIG. 7 illustrates an optical system where a compound lens is shown as an optical objective 115 forming an image 108 having the characteristic size 109. The object 100 has characteristic dimension dₒ(101), and it is assumed to be a size of the biggest object that can be imaged in a given configuration. In such cases, the characteristic dimension dₒ(101) defines the field of view (FOV). The object is formed at a distance zₒ (102) and the image is formed at a distance zᵢ (113).

The optical objective is characterized by its optical focal length Fₒ(104). One skilled in the art appreciates that manufacturers of optical objectives use several different characteristics to describe the objectives. In addition to the optical focal length 104, one can also specify the optical back focal length 107 as the distance between the last optical element of the objective and the image. Moreover, several manufacturers use the term ‘focal length’ for the back optical focal length, which might lead to an ambiguity. The term ‘optical focal length’ 104 used herein is understood in its classical form as depicted in FIG. 5. The distance between the object and the front element of the objective is called working distance 102 (see FIG. 6).

The formula (5) also holds for a compound lens such as an optical objective 115. Additionally, and angular field of view AFOV (103) is defined as:

$$A F O V = 2 \times {tan}^{- 1} \frac{{d}_{o}}{2 \times {z}_{o}} \qquad ( 6 )$$

or equally

$$A F O V = 2 \times {tan}^{- 1} \frac{{d}_{i}}{2 \times {z}_{i}} \qquad ( 7 )$$

which implies that

$$\frac{{d}_{i}}{{z}_{i}} = \frac{{d}_{o}}{{z}_{o}} \qquad ( 8 )$$

Magnification MAG is therefore given by the following formula:

$$MAG = \frac{{d}_{i}}{{d}_{o}} \qquad ( 9 )$$

Formulas (4), (5) and (8) can be combined to determine the size the object dₒ(FOV) and its location at a given image size dᵢ and an optical focal length Fₒ for a given image sensor and optical objective.

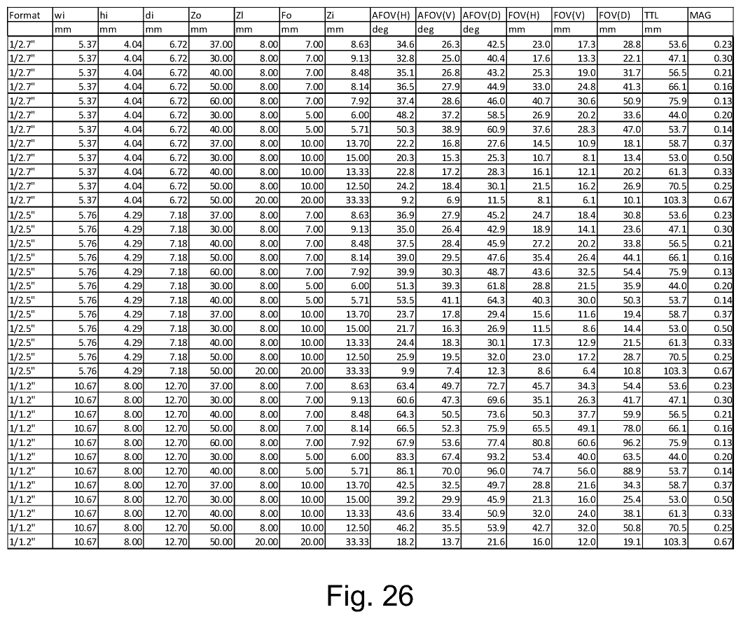

In several embodiments, both the object 100 and the image 108 are of a rectangular shape characterized by the height, the width, and the diagonal. A characteristic dimension of an object and an image (101 and 109, respectively) might be either a width, a height, or a diagonal, therefore one can define a vertical FOV(V), a horizontal FOV(H), and a diagonal FOV(D). FIG. 26 presents several preferable configurations of the embodiments disclosed herein.

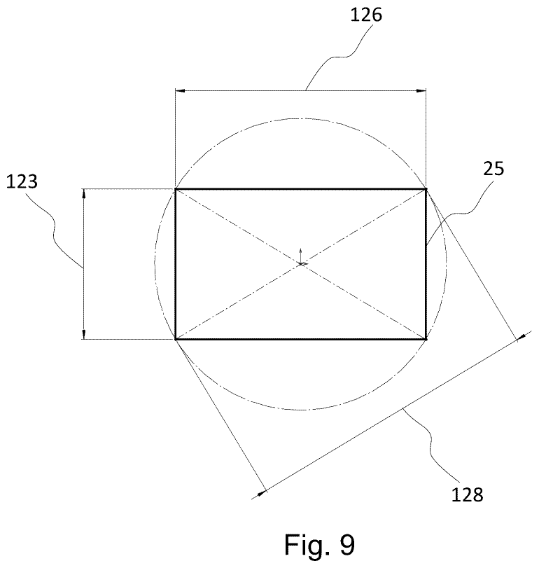

FIG. 9 illustrates major parameters of an image sensor. One skilled in the art appreciates that in practice the image is formed on a surface of an image sensor. Accordingly, the image plane can be regarded as a surface of an image sensor, and the parameters of an image such as the height 123, the width 126, and the diameter 128 are directly translatable into parameters of an image sensor 25. Moreover, in several embodiments, the size of an image is determined by the size of an image sensor. In some embodiments, the image sensor can be a CCD (Charge Coupled Device) camera, in some other embodiments it is an active-pixel sensor CMOS or any other image sensing device.

Image sensor devices have an optical format such as the typical 4:3 or 16:9. Several embodiments disclosed herein take advantage of image sensors of optical format 1/1″, 1/2″, 1/2.3″, 1/2.5″, 1/2.7″, 1/2.9″, 1/3″, 1/4″, 1/10″ or any other optical format.

FIG. 26 gives examples of embodiment configurations including an image sensor optical format an object size, and its location for a given optical objective.

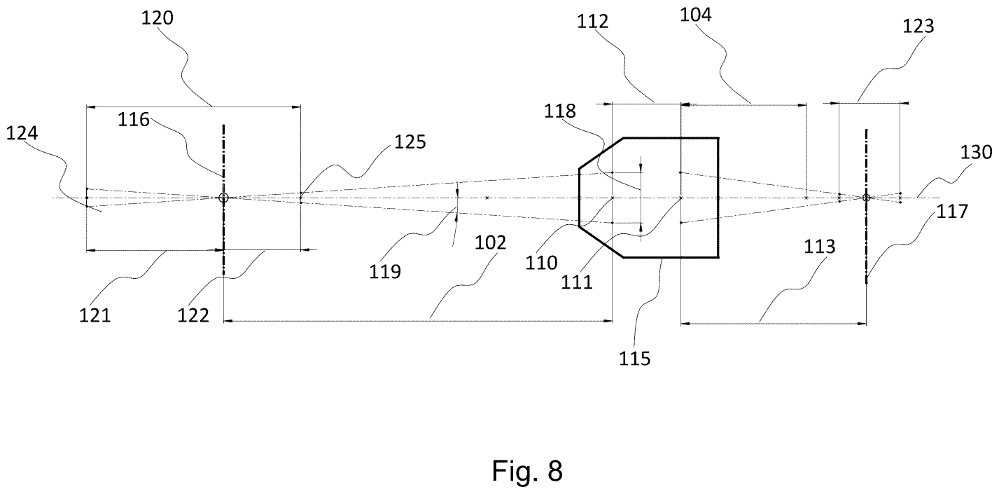

Some embodiments disclosed herein use an optical system like the one illustrated in FIG. 8. There are certain limitations for a given configuration of the objective with regards to the imaging capabilities, in particular, the resolution, the optical depth of field 120 and the optical depth of focus 123. The optical depth of field is a range along the principal optical axis 130 within which an object still appears sharp. The optical depth of focus is the distance along the principal optical axis 130 within which the image still appears sharp.

Diffraction is the limiting factor of the imaging capabilities of an optical system like the one depicted in FIG. 8. Diffraction limits the resolution, which is defined as the minimal distance between two distinct points of an object that can still be resolved in an image. The resolution is intrinsically linked to the numerical aperture NA, the light wavelength as well as the medium which light passes through. Numerical aperture is defined in equation 10 as follows:

$$N A = n sin α \qquad ( 10 )$$

where n is the refractive index of the medium, and a is the aperture half-angle 119 as depicted in FIG. 8.

Then the resolution is determined by the Rayleigh criterion and is given by:

$$RES = \frac{0.61 \times λ}{N A} \qquad ( 11 )$$

and the optical depth of field:

$$D O {F}_{o} = \frac{n \times λ}{N {A}^{2}} \qquad ( 12 )$$

where λ is the light wavelength.

In some embodiments, the resolution of the optical system in the device is in the range from 100 nm to 1000 μm. In some other embodiments, the resolution is in the range from 0.1 um to 100 μm. In more preferable embodiments, the resolution is in the range from 0.5 μm to 50 μm.

As part of the optical system or in addition to the optical system, embodiments may use light to illuminate the surface to be observed. Several embodiments disclosed herein use light with a wavelength in a range from 420 nm to 900 nm. Other embodiments use light with a wavelength from 100 nm to 20000 nm. In preferred embodiments that illuminate the observation area, the wavelength of the light is in a range from 420 nm to 890 nm.

Optical systems are also characterized by an optical F-number, describing the amount of light that passes through the objective. The optical F-number is defined in equation 13 as follows:

$${F}_{\#}^{o} = \frac{{F}^{o}}{{D}^{o}} \qquad ( 13 )$$

where D^{o} is the optical aperture diameter 118, illustrated in FIG. 8. In some embodiments, the optical F-number is in the range from 1 to 50. In some other embodiments, the optical F number is in the range from 1 to 20. In preferred embodiments, the optical F number is in the range from 1 to 5.

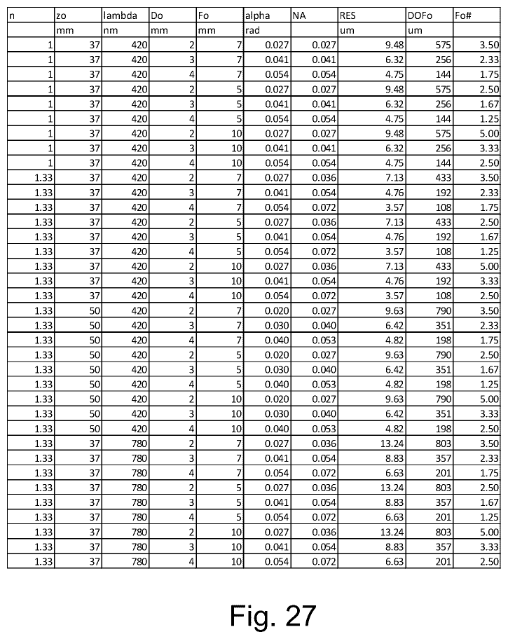

FIG. 27 presents several configurations of preferred embodiments outlining resolution, field of view and optical depth of field.

### Imaging Through Media of Variable Refractive Index

In some of the embodiments disclosed herein, the image is formed by light passing through one, or more than one medium, of a given refractive index. In some embodiments, the light travels through air. In some other embodiments, the light travels through water and air. In still yet other embodiments, the light travels through water, glass, and air. In some embodiments, the light travels through one or more media that can be a combination in any order of air, water, liquid, fluid, gas, plastic, polymer, and any optically transparent material for the given light wavelength range. The optically transparent material may be, for example, polycarbonate, Poly(methyl methacrylate), acryl, glass, or any other optically transparent material.

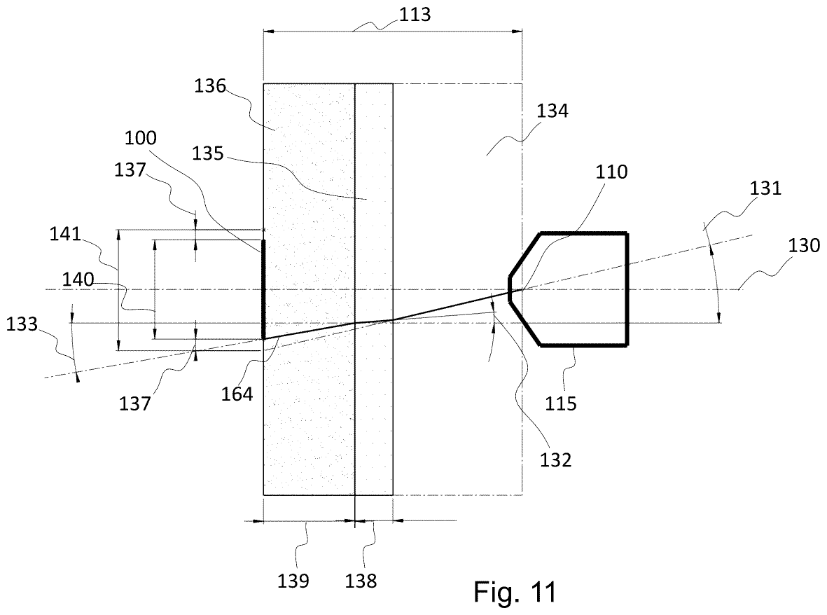

FIG. 11 illustrates a ray of light 164 emitted from the object 100 passing through medium 3 136 of refractive index n=n3 at angle 133. The ray is then refracted at the interface between medium 3 and medium 2 and it is traveling through medium 2 138 of refractive index n=n2 at angle 132. The ray is then being refracted again at the interface between medium 2 and medium 1 of refractive index n=n1 134. Ultimately, the light reaches the objective at angle 132. Given that n1<n3<n2, the effective angular FOV is reduced as angle 137 is smaller than the angular FOV of the objective 132. This also results in the FOV 140 that is smaller than the FOV of a system where medium 2 and medium 3 are the same as medium 1.

Some embodiments illustrate an optical system configured like the one depicted in FIG. 11. Medium 3 is water with n=1.33, with a thickness 139. The thickness of medium 3 may be any thickness but in this embodiment, the thickness 139 is 12 mm. Medium 2 is polycarbonate with n=1.53. Again, medium 2 may have any thickness but in this embodiment, medium 2 has a thickness of 5 mm. Medium 1 is air with n=1. Medium 1 may also have any thickness but in this embodiment, medium 1 has a thickness of 17 mm. The angular FOV of the objective is 26.5°. After including the refraction at the interfaces between medium 1, 2 and 3 the resulting angular FOV is reduced to 20.0° and the FOV is 13.76 mm, as opposed to 16.07 mm in case when the medium was just air.

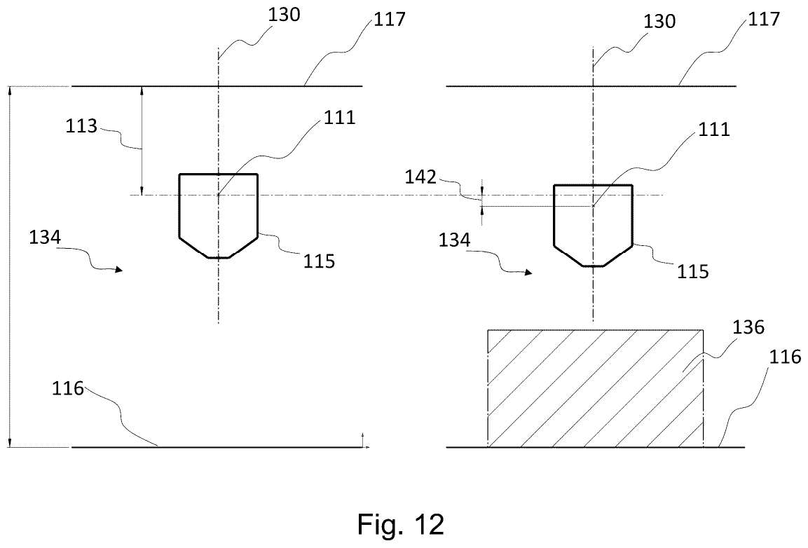

In several of the embodiments disclosed herein, the reduction of the FOV due to light passing through media of different refractive index makes the image appear ‘closer’ to the objective. Accordingly, the objective needs to be moved closer to the object by a distance 142 to produce a sharp image, as illustrated in FIG. 12. The adjustment is in the range from 1 μm to 1500 μm. In more preferable configuration, the adjustment is from 5 μm to 500 μm.

Acoustic Device with Adjustable Penetration Depth and Optical Imaging at Constant Optical Focal Length

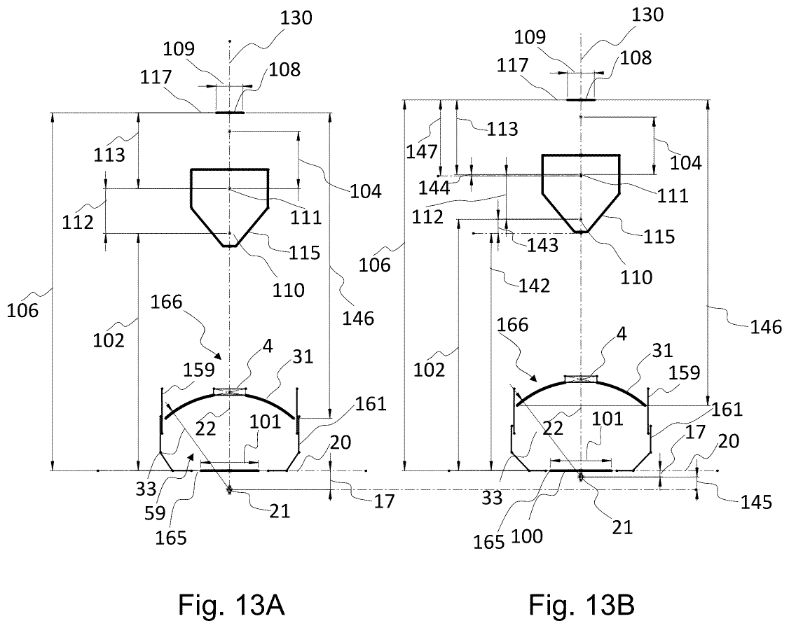

FIG. 13A and FIG. 13B illustrate an example of an embodiment comprising a focused acoustic source with a transparent optical path and an objective forming an image of the surface of the tissue to be treated (e.g. skin). The embodiment shown in FIG. 13A comprises a focused acoustic source 31 coupled to a fixed part of a housing 159. The focused acoustic source 31 has a hole. The embodiment shown in FIG. 13A further comprises an optical objective 115, which forms an image 108 of an object 100. The fixed part of the housing together with the adjustable part of the housing 161 form a housing that can hold an acoustically and optically transparent coupling medium 58. The adjustable part of the housing 161 comprises of one or more components and is configured to adjust the distance between the acoustic focal point 21 and the reference plane 20.

In some embodiments, the optical principal axis 130 is coaxial with the main acoustic axis 22. The optical system may be comprised of one or more lenses of several different shapes and sizes, e.g. convex-convex, plano-convex, convex-concave, concave-concave. The focused acoustic source 31 with a hole allows light to pass through the interior of the housing which makes continuous, real-time observation of the object 100 possible. As may be appreciated, the object 100 is preferably the treatment surface. In several embodiments disclosed herein, the object 100 may be, but is not limited to, a surface of the skin, a surface of a tissue, a surface of the skin with a tumour, and numerous other surfaces. In several embodiments, the adjustable part of the housing 161 features an opening 165 that allows light and acoustic waves to pass through. The opening is covered with an optically transparent and acoustically transparent material, called the acousto-optic window 168. In some embodiments, this material can be a thin plastic foil, for example but not limited to low density polyethylene. The hole in the focused acoustic source is filled with an optical separator 4. The optical separator 4 functions as a plug fabricated from an optically transparent material, blocking the hole in the acoustic radiator, so it prevents the coupling medium 58 from leaving the impermeable chamber 166, providing however the optically transparent path for the observation. In some embodiments, the separator can be directly bonded to the acoustic source 31. In, some other embodiments, it is bonded to specific elements of the fixed part of the housing 159. The optical separator may be fabricated from polycarbonate, plexiglass, transparent plastics, glass or other transparent materials.

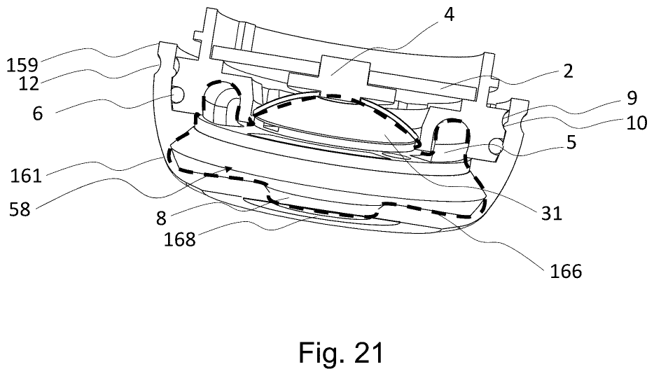

In several embodiments the fixed part of the housing 159, the adjustable part of the housing 161, the focused acoustic source 31, optical separator 4, and the opto-acoustic window 168 are configured to form a watertight or impermeable chamber 166, as depicted in FIG. 21.

In some embodiments, the optical objective 115 together with the watertight chamber 166 are configured to allow for imaging of the object coupled to the opening in the adjustable part of the housing. The primary principal point of the objective 110 is at the distance 102 from the reference plane 20 being also the object plane. The image is at the distance 113 from the secondary principal point of the objective 111.

In some of the embodiments disclosed herein, the acoustic device is configured to allow for adjustment of the penetration depth. In some embodiments, the change of the penetration depth also changes the total optical track length 106 resulting in defocusing of the image 108. In some embodiments, the image can be brought back to focus by adjusting the distance 113 along the longitudinal axis between the objective 115 and the imaging plane 117, given that the optical focal length of the objective remains constant.

Given the following system of equations:

$$\{ \begin{matrix}z = {z}_{i} + {z}_{o} + {z}_{l} \\ {F}_{o} = \frac{{z}_{i} \times {z}_{o}}{z - {z}_{1}}\end{matrix} \qquad ( 14 )$$

where z is the total optical track length, zₗ is the length of the objective, zᵢ and zₒ are distances to image and object respectively and Fₒ is the optical focal length, the following formula can be used to estimate the new zᵢ* after a change of z due to penetration depth adjustment resulting in new total optical track length z*:

$${z}_{i}^{*} = 1 / 2 ( \sqrt{( {z}_{1} - z \text{*)} ( 4 {F}_{o} - z * + {z}_{l} )} + z * - {z}_{l} ) \qquad ( 15 )$$

Returning to FIG. 13A and FIG. 13B, the embodiment shown in FIG. 13A has an optical focal length Fₒ of 9 mm, and zₗ=7 mm. The acoustic focal length is 15 mm, and the diameter of the acoustic focusing source is 20 mm, and the operating frequency of the focused acoustic source is 12 MHz. The embodiment is configured to produce a penetration depth of 3 mm. The resulting zᵢ=11.88 mm, zₒ=37.12 mm and the total optical track length is 56 mm.

The same embodiment is re-configured to produce a penetration depth of 1 mm, as shown in FIG. 13B. This is achieved by adding 2 mm distance to the adjustable part of the housing 161 or by moving the adjustable part of the housing by 2 mm away from the focused source 31. After the re-configuration the total optical track length is 58 mm, and the resulting zᵢ=11.67 mm, zₒ=39.33 mm. The objective is thereby moved towards the image plane by 0.21 mm to bring the image back to sharpness.

Acoustic Device with Adjustable Penetration Depth and Optical Imaging at Variable Optical Focal Length

In some different embodiments, the adjustable optical focal length is provided by one or more lenses of adjustable optical focal length. In such case the re-focusing of the image after adjustment of the penetration depth 17 does not require a mechanical movement of the objective, keeping the image distance to the secondary principal point of the objective 113 constant for all ranges of the penetration depth. In many cases it is advantageous due to simplicity, low cost and compactness. In some embodiments, one or more of the lenses in the objective have the possibility of changing their optical focal length. In some embodiments it is achieved by use of liquid lenses that can change their shape and therefore, change their radius of curvature resulting in the change of the optical focal length. In some embodiments, the adjustable optical focal length lenses are electrowetting, use of shape-changing polymers and acousto-optical tuning, where the optical focal length is controlled by an electric signal.

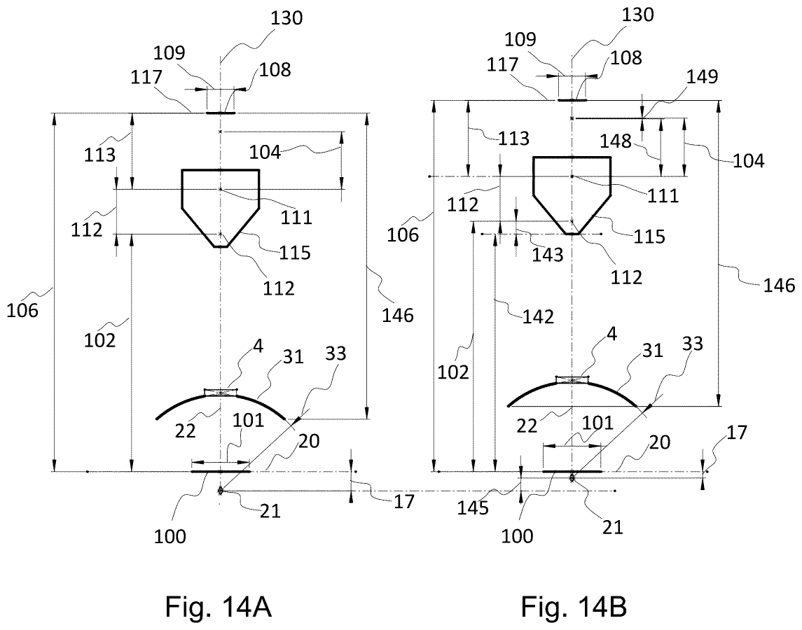

As an example, an embodiment may be configured as shown in FIG. 14A with an optical focal length Fₒ of 9.00 mm, and zₗ=7 mm. The objective comprises at least one adjustable optical focal length lens using electrowetting. The acoustic focal length is 15 mm, the diameter of the acoustic focusing source is 20 mm and the operating frequency of the focused acoustic source is 12 MHz. The embodiment is configured, e.g. through use of the appropriate adjustable part of the housing to produce the penetration depth of 3 mm. The resulting zᵢ=11.88 mm, zₒ=37.12 mm and the total optical track length is 56 mm.

The same embodiment is re-configured to produce penetration depth of 1 mm, e.g. by adding 2 mm distance to the adjustable part of the housing (FIG. 14B). The total optical track length is now 58 mm (56 mm+2 mm), the image to secondary principal point distance remains unchanged zᵢ=11.88 mm, and zₒ=39.12 mm. The objective is kept at the same original position; however, its optical focal length is adjusted to be equal to Fₒ=9.11 mm in order to refocus the image after reconfiguration.

Acoustic Device with Adjustable Penetration Depth and Fixed Optical Imaging System of Extended Optical Depth of Field

Some other embodiments disclosed herein comprise a focused acoustic source 31 coupled to a fixed part of a housing 159. The focused acoustic source 31 has a hole. The embodiment further comprises an optical objective 115, which forms an image 108 of an object 100. The fixed part of the housing together with the adjustable part of the housing 161 form a housing that can hold an acoustically and optically transparent coupling medium 58. The adjustable part of the housing 161 comprises of one or more components and is configured to adjust the distance between the acoustic focal point 21 and the reference plane 20. In these embodiments, the optical imaging system is configured so it produces a sharp or acceptably sharp image at all attainable penetration depths of the system, i.e. even after the penetration depth is changed. It may be achieved by use of objectives of extended (relatively large) optical depth of field 120. In some embodiments the optical depth of field 120 is matched (equal or larger than) with the attainable range of penetration depths. In some embodiments the optical depth of field is 0 mm to 5 mm or 0.1 mm to 5 mm. In some other embodiments, the optical depth of field is 1 mm to 100 mm. In preferable embodiments, the optical depth of filed is 1 mm to 10 mm.

Acoustic Device with Adjustable Penetration Depth an Optical Imaging in Non-Coaxial Arrangement

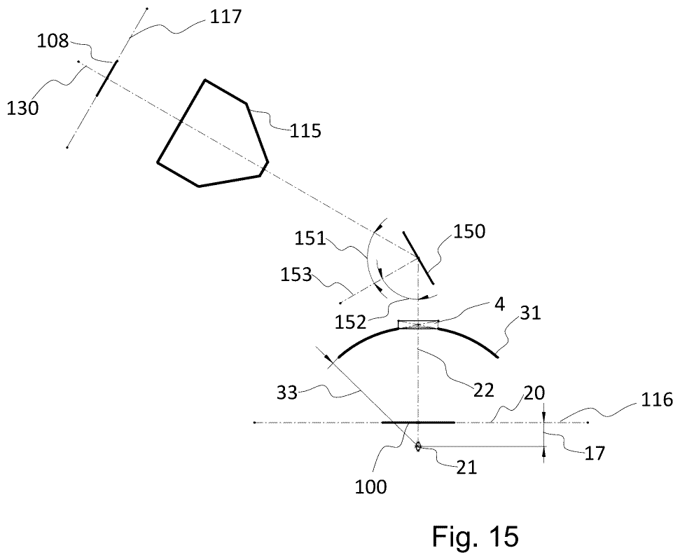

FIG. 15 illustrates an example of an embodiment comprising a focused acoustic source with a transparent optical path and an objective forming an image of a surface of the tissue to be treated (e.g. skin) where a portion of the optical main axis of the optical objective direction is not coaxial with the acoustic main axis. Such a configuration is advantageous because it allows for a more flexible arrangement of acoustic and optical devices. In some embodiments, the light traveling along the optical principal axis 130 that is coaxial with the acoustic main axis 22 is reflected by an optical reflective device 150, e.g. mirror passed the separator 4, and it is directed at a reflection angle 151 into the optical system comprising but not limited to a system of lenses, or objectives 115. In some embodiments, the optical axis is reflected or refracted more than one time using a combination of, but not limited to, optical devices such as mirrors, prisms, splitters, etc. Moreover, the transparent path can be split into more than one path that is then directed into different optical systems for further processing by using, but not limited to, image sensors, eyepiece oculars, etc. In such embodiments, the image plane(s) 117 are not parallel to the object plane 116.

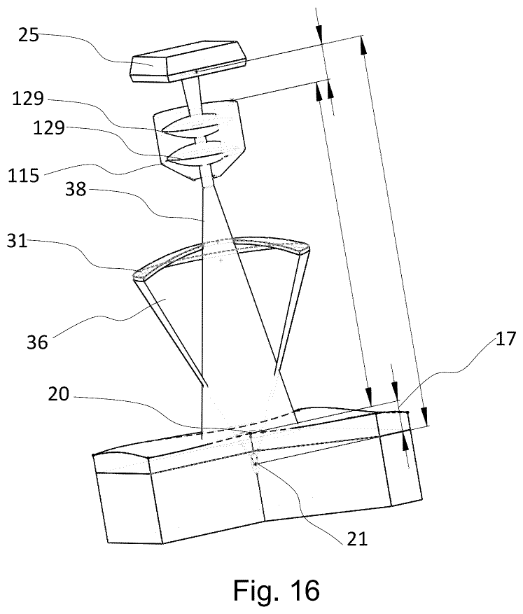

Preferred embodiments disclosed in here comprise optical devices providing continuous and real-time observation capabilities of the surface of the tissue to be treated (e.g. skin). The acoustic field 36 generated by the focused acoustic source 31 and the optical field 38 provided by the objective 115 are combined which is schematically illustrated in FIG. 16.

### Bubble Trap

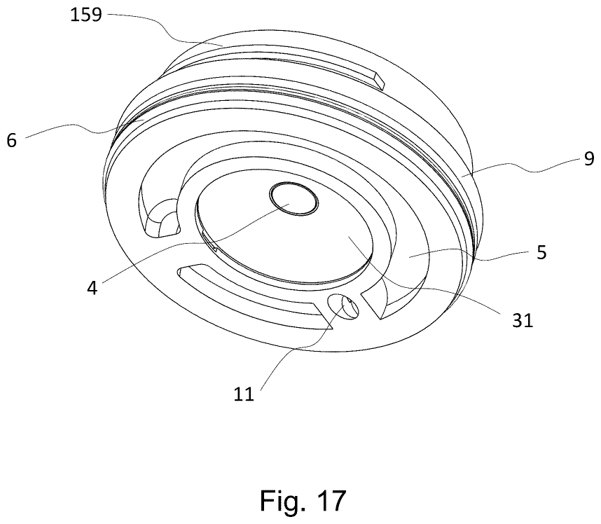

FIG. 17 illustrates an isometric view of a focused acoustic source 31 coupled to the fixed part of the housing 1. The embodiment shown in FIG. 5, comprise a focusing acoustic source 31 coupled to the housing 1 that includes a bubble trap 5. In some embodiments, gas (e.g. air) dissolved in the coupling medium 58 (e.g. water) forms bubbles that can obstruct the clear view of the object, such as the surface of the skin. Moreover, the bubbles may block the acoustic waves travelling from the focused acoustic source to the tissue. Accordingly, it is proposed herein that a bubble trap 5 is designed into the inside cavity holding the coupling medium 58 to reduce or eliminate bubbles during operation. In some embodiments the bubble trap is a groove or channel that is located around the circumference of the focused acoustic source 31 in such a way, so the chamber formed by the groove is located above the line of the focusing acoustic source 31. In some embodiments the bubble trap is round (see FIG. 17), but in other embodiments it is square, rectangular and of any other shape.

The distance between the base of the bubble trap and its ceiling is called the depth of the bubble trap 167. In some embodiments the depth of the bubble trap is from 0.1 mm to 100 mm. In some other embodiments, the depth of the bubble trap 167 is 0.5 mm to 20 mm. In more preferable embodiments, the depth of the bubble trap 167 is in the range from 1 to 10 mm. Some embodiments feature the bubble trap 5 coated with a coating attracting and holding bubbles such as bubble-capturing surfaces, nanoparticle-based coating, etc. Non-limiting examples of such coatings include parylene coating, polyamide coatings, etc.

During operation, bubbles tend to aggregate around the apex of the focusing piezoelectric component 31 on its concave side, where the optical separator 4 is located. Having the bubbles aggregate around the apex of the focusing piezoelectric component 31 on its concave side amplifies the undesired effect because the transducers/handpieces are typically operated facing down. The bubble trap 5 has a form of a groove or a channel or a vessel that spans around the acoustic source allowing the bubbles to be transferred from the undesired locations to the bubble trap 5 through a simple operation of turning the handpiece/transducer upside-down and then slowly returning it to the desired position (facing down) with optional gentle shaking while the bubbles get trapped in the bubble trap 5. The bubbles stay in the trap due to gravity.

The bubble trap may be a continuous channel the circumvents all the way around the piezoelectric component 31. However, in other embodiments, the bubble trap may be comprised of more than one channel such as the example shown in FIG. 17. In FIG. 17, two channels are used that surround the piezoelectric component 31. In other embodiments, more than two channels may be used.

An Acoustic Device with Adjustable Penetration Depth in Increments

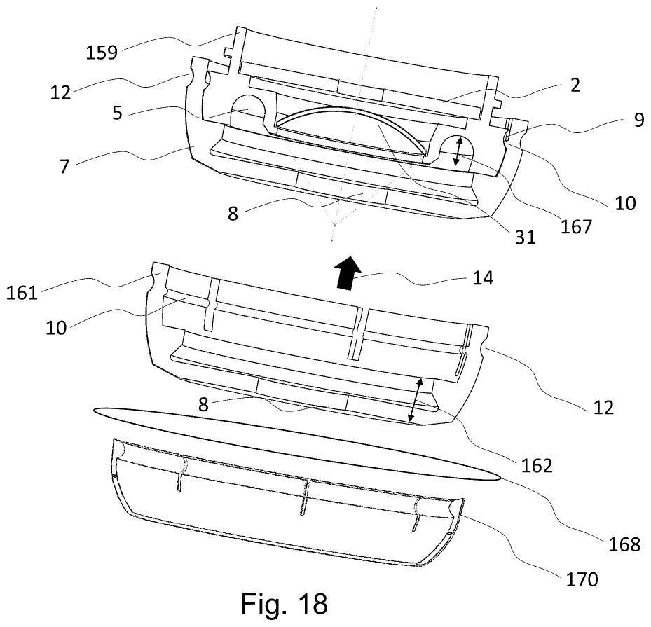

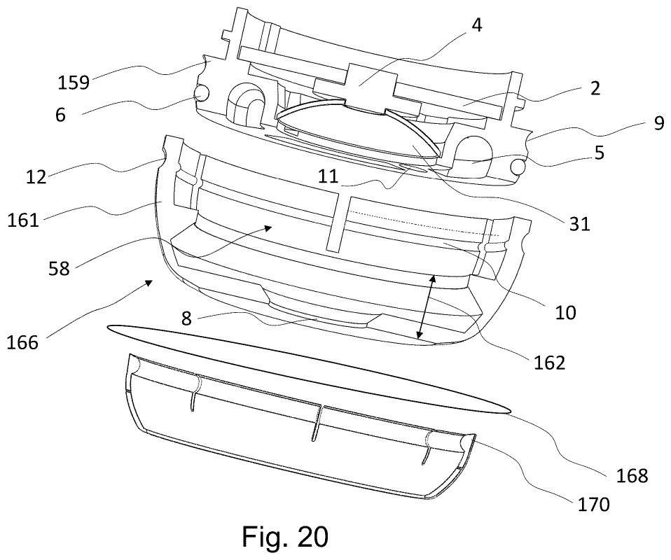

As shown in FIG. 18 and FIG. 20, and previously in FIG. 4, in some of the embodiments disclosed herein, the penetration depth adjustment is performed in steps predefined by the length of the adjustable part 162. One fixed housing element 159 is combined with several adjustable housing elements 161, defining a range of penetration depths. The adjustment is performed in the following steps: the fixing collar 170 is released, the acousto-optic window 168 is removed, the coupling medium 58 is removed through a re-fill port, the adjustable part of the housing 161 is replaced with the new one of the desired penetration depth, coupling medium 58 is refilled through the re-fill port, the opening 8 is covered by an acousto-optic window 186, the acousto-optic window is affixed back onto the housing using the fixing collar 170 and fixing groove 12.

In several embodiments disclosed herein the opening 8 is used as the re-fill port. In some other embodiments, a separate, and/or plurality of, re-fill ports is/are provided.

In some embodiments, the adjustable range is from 0.1 mm to 1000 mm. In some other embodiments, the adjustable range is from 0.2 mm to 100 mm. In more preferable embodiments, the adjustable range is from 0.2 mm to 10 mm. The steps of the range can vary and may be in steps of 0.1 mm to 100 mm. In some other embodiments the adjustment step is matched with the acoustic depth of focus so it is a fraction or multiplier of the acoustic depth of focus in the range from 0.01 to 50.

In some embodiments the adjustable housing element 161 is affixed into the fixed housing element 159 using a tight fit (snap-on, clip-on) with a groove 9 and a ridge 10. FIG. 18 shows such a configuration where the direction of affixing 14 is indicated as well.

In many embodiments the coupling between the fixed and adjustable part of the housing is achieved but not limited to a snap-on, threaded, tight fit, press fit, etc.

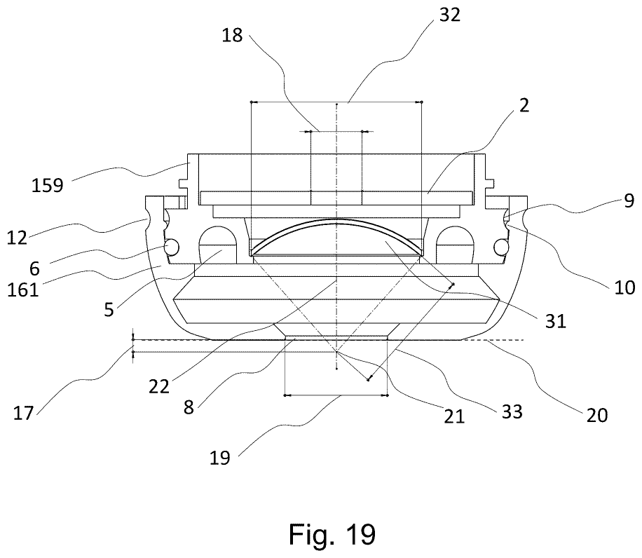

FIG. 19 illustrates a cross-section of an example of a focused acoustic source coupled to the fixed part of the housing coupled with an adjustable part of the housing. The coupling between the fixed and adjustable part of the housing is made watertight using a seal 6. Some embodiments disclosed herein comprise a watertight coupling between the fixed housing element 159 and adjustable housing element 161. In many embodiments, the watertightness is provided by a solid, or liquid sealant. As shown in FIG. 19, in some embodiments, the watertightness is provided by one or more seals 6. The seal may also be an O-ring or other type of seal with various different cross-sections. The seal may be made from silicone, plastic, polymer, rubber or various different other materials.

In preferred embodiments, the adjustable part of the housing 161 includes an opening 8 providing the clear path for acoustic waves toward the acoustic focus point 21. In some embodiments, the opening is round and has a diameter 19 in the range of 0.1 mm to 100 mm. In other embodiments, the round opening is in the range from 1 mm to 30 mm. In some other embodiments, the opening may be a square or rectangular shape, or any other shape.

In preferred embodiments disclosed in here the electric matching circuit 2 is provided to aid the energy flow between the RF power amplifier 308 and the associated output filters and matching 309 and the focused acoustic source 31. In preferred configurations the electric matching circuit provider a real and pre-defined impedance at the selected frequency or frequency range. In more preferable configurations the impedance is matched to 1 to 1000 Ohm at single or multiple frequencies from the between 1 kHz and 1000 MHz, in more preferable it is matched to 50 Ohm in frequency range from 1 MHz to 100 MHz. In some embodiments the matching circuit is placed onto single or multipole printed circuit boards having one or more openings providing clear optical path along the optical axis 130. The diameter of the opening in the matching circuit 18 is in the range from 1 to 100 mm.

An Acoustic Device with Optical Observation and Adjustable Penetration Depth in Increments

As discussed above with respect to FIG. 4 and FIG. 18 and now here with respect to FIG. 20, in several embodiments disclosed herein, the penetration depth adjustment is performed in steps predefined by the length of the adjustable part 162. The focused acoustic source is coupled to the fixed housing element 159 and is combined with one of a plurality of adjustable housing elements 161, defining a range of penetration depths. The penetration depth may be changed by simply changing the adjustable housing element 161 from the current one to one of the other adjustable housing elements in the plurality of adjustable housing elements.

As shown in FIG. 21, the fixed housing part 159 together with the adjustable housing part 161, the focusing acoustic source 31, optical separator 4 and the opto-acoustic window 168 form a watertight chamber 166 holding the coupling medium 58.

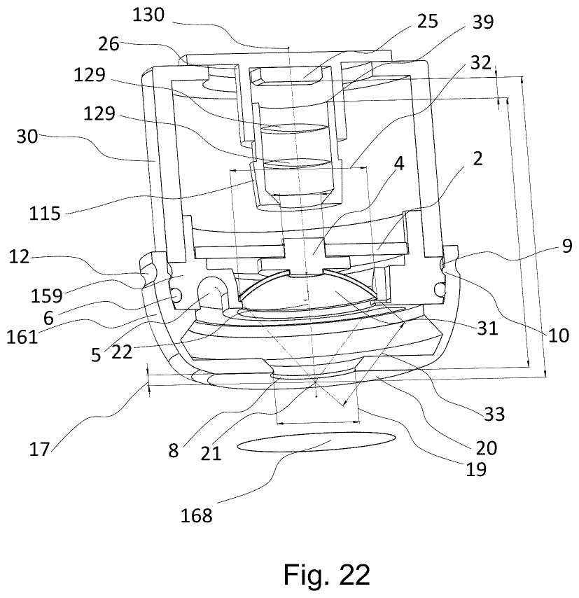

FIG. 22 illustrates an isometric three-dimensional section view of an acoustic device with an adjustable penetration depth and an optical observation. As may be seen in FIG. 22, the focused acoustic source 31, regardless of whether it is constructed as a single focused piezoelectric element, a multielement component, or by a planar element with an attached element used to focus the acoustic signal, may be manufactured with a hole. In preferred embodiments, the hole is in the center of the focused acoustic source 31 along the main acoustic axis 22. As may be appreciated, in the embodiment shown in FIG. 22, the acoustic axis 22 is also the longitudinal axis of the device. The hole allows for mechanical stress relive during high-power operation of the focused acoustic source 31. In addition, the hole may be used to allow for optical access of the imaging system through fitted optical separator 4.

In preferable embodiments, the fixed part of the housing 159 is coupled to the focused acoustic source 31 and is used together with a range the adjustable part of the housings 161 of different length 162 so the penetration depth 17 is adjustable in increments. In some embodiments the increment is in the range from 0.1 mm to 100 mm, in some other embodiments it is in the range from 0.2 to 5 mm. In addition, the fixed part of the housing is coupled to the handpiece housing 30 comprising the camera housing 26 containing an image sensor 25 and a movable objective 115. The camera fixture 26 allows for the motion of the objective towards and from the image sensor. One skilled in the art appreciates that an image sensor 25 is placed so its sensing surface coincides with the image plane 117 of the imaging system, as described earlier. Moreover, the object being observed is the surface of a tissue to be treated, e.g. the surface of the skin. The surface may be observed once the reference plane of the housing 20 is coupled to the surface of the tissue to be treated.

Many embodiments disclosed herein provide the adjustment of the penetration depth 17 by provision of a number of adjustable parts of the housing 161. One skilled in the art appreciates that each change of the penetration depth changes the distance of reference plane 20 which in the preferred configuration is the same as the object plane 116 to the image plane 117, which in the preferred configuration is the sensing surface of the image sensor 25. In some embodiments this means that the objective needs to be moved along the optical principal axis 130 in order to refocus the image. Formulas (4) to (8) can be used to estimate the necessary displacement.

In some other embodiments the optical system can be designed so its optical depth of field covers the range of required penetration depth changes. In some embodiments the optical depth of field is 100 mm, in some other it is 5 mm.

An Acoustic Device with Optical Observation and Continuously Adjustable Penetration Depth

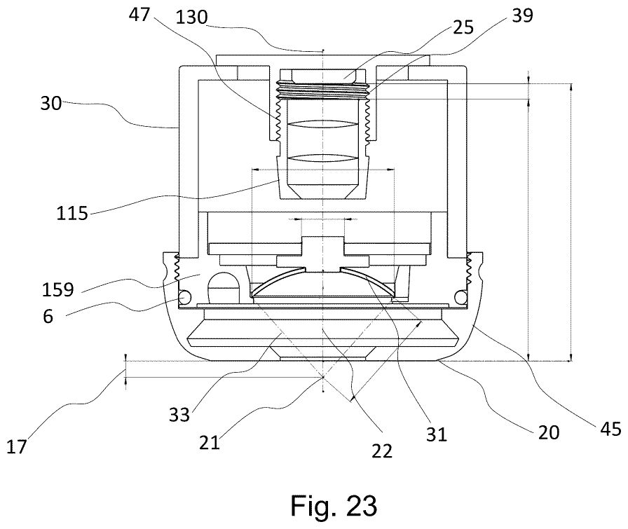

FIG. 23 illustrates a cross-section view of an acoustic device with continuously adjustable penetration depth and an optical observation. As may be seen in FIG. 23, the focused acoustic source 31, regardless of whether it is constructed as a single focused piezoelectric element, a multielement component, or by a planar element with an attached element used to focus the acoustic signal, may be manufactured with a hole. In preferred embodiments, the hole is in the center of the focused acoustic source 31 along the main acoustic axis 22. As may be appreciated, in the embodiment shown in FIG. 23, the acoustic axis 22 is also the longitudinal axis of the device. The hole allows for optical access of the imaging system through fitted optical separator 4.

In preferable embodiments the fixed part of the housing 159 is coupled to the focused acoustic source 31 and is used together with a continuously adjustable part of the housings 45 so the penetration depth 17 is continuously adjustable. In some embodiments the range of adjustment is from 0 mm to 1000 mm, in some other embodiments it is in the range from 0 to 20 mm. In some embodiments the continuous adjustment is provided by a threaded coupling 46 so the rotation of the adjustable part of the housing around the main acoustic axis 22 provides the relative motion of the adjustable part of the housing 45 along the main acoustic axis 22.

In addition, the fixed part of the housing is coupled to the handpiece housing 30 comprising the camera housing 26 containing an image sensor 25 and a movable objective 115. The camera fixture 26 allows for the motion of the objective towards and from the image sensor. One skilled in the art appreciates that an image sensor 25 is placed so its sensing surface coincides with the image plane 117 of the imaging system, as described earlier. Moreover, the object is the surface of a tissue to be treated, e.g. the surface of the skin, once the reference plane of the housing 20 is coupled to the surface of the tissue to be treated.

Many embodiments disclosed herein provide the adjustment of the penetration depth 17 by provision of continuously adjustable housing 45. One skilled in the art appreciates that each change of the penetration depth changes the distance of reference plane 20 which in the preferred configuration is the same as the object plane 116 to the image plane 117, which in the preferred configuration is the sensing surface of the image sensor 25. In some embodiments this means that the objective needs to be moved along the optical principal axis 130 in order to refocus the image. Formulas (4) to (8) can be used to estimate the necessary displacement. In some embodiments the motion of the objective 115 is provided by a threaded coupling 47.

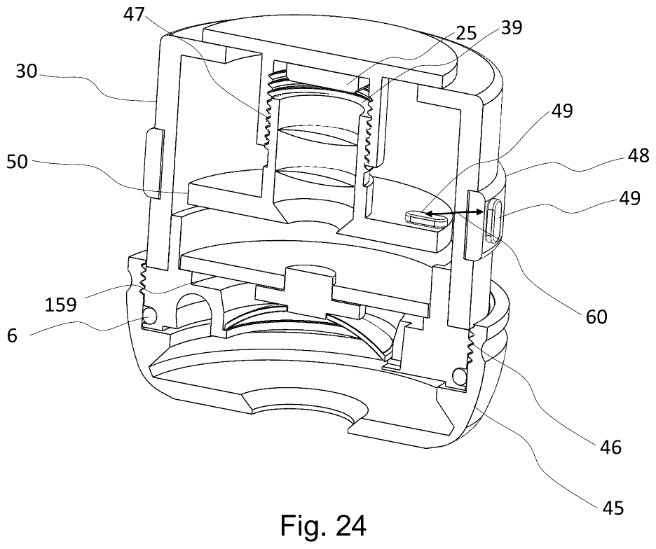

FIG. 24 illustrates an isometric three-dimensional section view of an acoustic device with continuously adjustable penetration depth and an optical observation. Similar to previous embodiments, the embodiment in FIG. 24 comprises a focused acoustic source 31 with a hole in the center along the main acoustic axis 22. The hole allows for optical access of the imaging system through fitted optical separator 4.

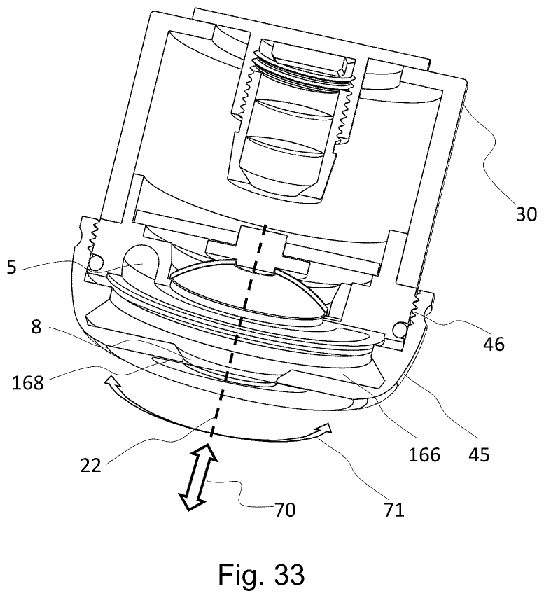

In preferable embodiments, the fixed part of the housing 159 is coupled to the focused acoustic source 31 and is used together with a continuously adjustable part of the housings 45 so the penetration depth 17 is continuously adjustable. In some embodiments the range of adjustment is from 0 mm to 1000 mm, in some other embodiments, it is in the range from 0 to 20 mm. In some embodiments, the continuous adjustment is provided by a threaded coupling 46 so the rotation 71 of the adjustable part 45 of the housing around the main acoustic axis 22 provides the relative motion 70 of the adjustable part of the housing 45 along the main acoustic axis 22, as illustrated in FIG. 33. The motion is continuous and reversable; thus, the adjustment of the penetration depth is continuous and reversable. In some embodiments the pitch (the distance between corresponding points on adjacent threads) of the threaded coupling 46 is selected so it provides a precise control over the penetration depth through rotation along the direction 71. In some embodiments the pitch is 0.01 mm to 10 mm, in the preferred embodiment it is in the range 0.1 mm to 5 mm. The adjustment of the penetration depth changes the overall volume of impermeable chamber 166. In some embodiments, it will be required to remove or add the coupling medium 58 after adjustment, through one or more in-fill ports. The in-fill port can be the opening 8, and the re-fill of coupling medium can involve removal of the acousto-optic window 168. In the preferable embodiments, this is not required, the change in volume of the impermeable chamber 166 is compensated by the presence of a coupling medium buffer. In some embodiments the buffer is in the form of a vessel in a hydraulic connection with the impermeable chamber 166. In more preferable embodiments, the function of the coupling medium buffer is provided by the bubble trap 5. In some embodiments the adjustment is controlled by a scale engraved or printed on the outside of the housing on both the stationary part of the housing 30 and the adjustable part 45. In another embodiment the control of the penetration depth adjustment is aided by one or more distance or proximity sensor. The senor or plurality of sensors can employ any principle, it can be but is not limited to an electric field principle, magnetic field principle, optical, etc.

In addition, the fixed part of the housing is coupled to the handpiece housing 30 comprising the camera housing 26 containing an image sensor 25 and a movable objective 115. The camera fixture 26 allows for the motion of the objective towards and away from the image sensor.

In the embodiment shown in FIG. 23, the adjustment motion of the objective 115 is provided by a threaded coupling 47. In the preferred configuration the threaded fixture with a shoulder 50 is providing the magnetic link 60 with the external ring 48 through a system of two or more magnets 49. Turning the ring 48 allows for continuous adjustment of the optical imaging system so the image stays sharp at any penetration depth from the range 0 to 1000 mm.

### Additional Configurations

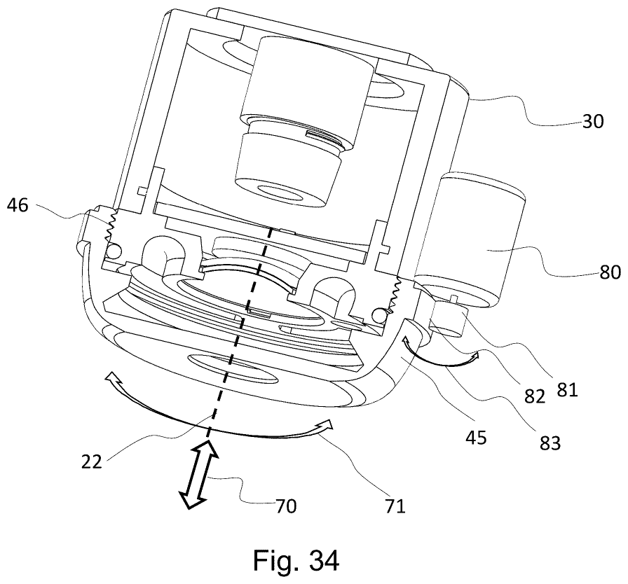

Several embodiments disclosed herein may comprise a motorised adjustment of the penetration depth through incorporation of one or more mechanical devices providing motion, such as motors, piezo motors, stepper motors, electromagnetic actuators, and other devices. In addition, the adjustment of the objective is also provided by one or more mechanical devices providing motion, such as motors, piezo motors, electromagnetic actuators, and other devices. In some other devices the optical focusing is provide by at least one lens of an adjustable optical focal length. FIG. 34 illustrates an embodiment disclosed herein where a rotor 81 of a motor 80 is mechanically coupled through a coupling 82 to the adjustable part of the housing 45. The housing of the motor 80 is affixed onto the stationary part of the housing 30. The rotation 83 of the rotor 82 is therefore translated into the rotation of the adjustable part of the housing 45 and through the threaded coupling 46 it provides the adjustment of the penetration depth in the direction 70 along the main acoustic axis 22. In some embodiments the mechanical coupling 82 is a friction-based coupling, in some other embodiments is gear-based coupling involving two or more gears. In yet another embodiment the motor 80 is equipped with a gear box reducing the rotation speed and increasing the torque, providing a precise adjustment of the penetration depth. In the preferred embodiments, the pitch of the threaded coupling 46 combined with the rotation speed of the motor 80 through the gear system is configured so it provides precise control of the penetration depth with resolution in the range of 0.001 mm to 100 mm, in yet another configuration the resolution is in the range of 0.01 to 1 mm. The pitch of the threaded coupling 46 is in the range from 0.01 mm to 100 mm, in more preferable configurations it is in the range from 0.1 to 10 mm. The rotational speed of the motor 80 is configured so it is in the range from 1 rpm (rotation per minute) to 10000 rpm. In more preferable configurations, the rotational speed of the motor is in the range form 10 rpm to 500 rpm.

Several embodiments may take advantage of auto focus algorithms that can be incorporated into optical re-focusing after adjustment of the penetration depth.

In some embodiments, the penetration depth can be adjusted continuously during the treatment according to the pre-scribed trajectory pre-determined by diagnostic methods such as ultrasonic imaging, magnetic resonance imaging, etc.

The real-time continuous imaging capability provided by the embodiments disclosed herein can be further used for image processing providing additional information to the operators. Some embodiments can be configured so the real-time image of the treated surface 200 is supplemented by a virtual overlay image 201 showing the locations of the treated points 205. In some embodiments, the overlay can display information about locations already treated and those that should be treated using different symbols, to aid the operator. Augmented reality algorithms, or other closed loop tracking algorithms, may be used to track the moving image of the surface of the skin. In some embodiments, the specific features of the skin, such as the natural texture 203 and location of hair 206 can guide the overlayed information.

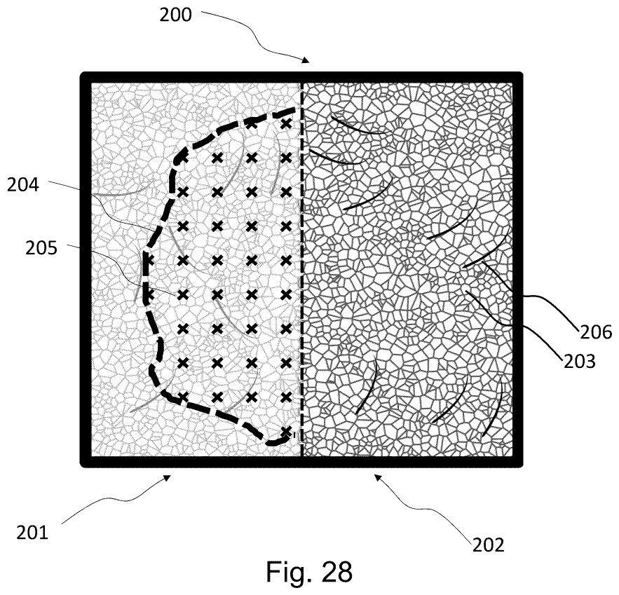

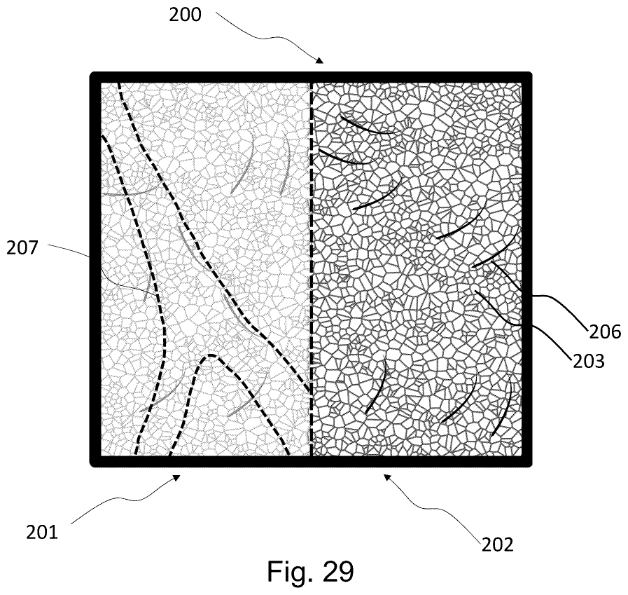

In some embodiments, the overlayed graphics contain the information regarding the treatment protocol, such as location of the POTs and/or virtual contour of the lesion 204, as illustrated in FIG. 28. Moreover, the virtual overlay information can be supplemented by data provided by other diagnostic modalities such as but not limited to ultrasonic imaging, magnetic resonance imaging, etc. FIG. 29 illustrates an example of virtual overlay showing an outline of the blood vessels detected using for example ultrasonic imaging. In some embodiments Artificial Intelligence algorithms are provided and implemented to aid the data fusion between the virtual overlay information and diagnostic systems.

### Acoustic Focusing Source

Generally speaking, any type of acoustic source 31 may be used in the embodiments described herein. In preferred embodiments, the focusing acoustic source 31 comprises a piezoelectric element 3 that has been fabricated into a section of a shell e.g. a sphere with a specific geometrical focal length, an element thickness defining its thickness resonance frequency and a diameter defining its aperture. In one embodiment, the focal length is in the range from 5 to 500 mm, and thickness of the element ranges from 0.01 mm to 20 mm, while the aperture is in the range from 10 mm to 1000 mm. In another embodiment, the acoustic focal length is in the range from 1 mm to 50 mm and thickness of the element ranges from 0.05 mm to 10 mm, while the aperture is in the range from 1 mm to 200 mm.

Preferably, the acoustic focusing source has a one or more holes that are fitted with an optical separator. In some embodiments, the hole is in the center of the focused source. In some other embodiments, the hole is in an off-center location. In many embodiments described herein, the hole in the focused acoustic source provides the clear optical path for optical observation of the surface of the tissue to be treated.

In a preferred embodiment, the focused acoustic source 31 may be a piezoelectric element with a hole 3. The piezoelectric element 3 may be made from a piezoelectric material such as doped lead zirconate titanate (PZT). PZT is a preferred choice because it has good energy conversion properties (high coupling coefficient k₃₃ and high value of d₃₃) and a relatively low cost.

In other embodiments, the acoustic source 31 is made from alternative piezoelectric materials such as, but not limited to, single crystals made from lithium niobate (LNb), aluminium nitride (AlN), lead magnesium niobate-lead titanate (PMN-PT) or quartz; polycrystal ceramic materials made from lead-meta-niobate, potassium sodium niobate (KNN), barium titanate (BaT), bismuth titanate (BT), bismuth sodium titanate (BNT), bismuth sodium titanate-bismuth titanate (BNT-BT); or polymeric materials made from polyvinylidene fluoride (PVDF).

In other embodiments, the focused acoustic source 31 can be replaced by an alternative active element, such as capacitive micromachined ultrasonic transducers (CMUTs), piezoelectric micromachined ultrasonic transducers (PMUTs) or similar.

In other embodiments, other materials may be used for the focused acoustic source 31 including combinations of materials and layers. In yet other embodiments, piezoelectric composites may be used for the focused acoustic source 31.

In some embodiments, where the focused acoustic source 31 is a piezoelectric element 3, the device has a mechanical quality factor (Qₘ) higher than 1000. In other embodiments, a Qₘ higher than 100 may be used.

In some embodiments, the focused acoustic source 31 is a planar element with an attached additional element (acoustic lens) used to focus the acoustic waves into a defined focal point 21. In some configurations, the lens is made from a low acoustic loss material characterised by the speed of sound that is lower than the speed of sound in coupling medium 58 (e.g. water). In this case the lens is of convex shape, typically made from a polymer, for example PDMS (Poly-dimethyl-siloxane), having typically 950 m/s speed of sound. In another configuration, the acoustic lens is of concave shape, being characterised by speed of sound that is higher than speed of sound in the coupling medium 58. This can be achieved by employing composite materials, such as polymer filled with metallic filler. In one embodiment, the polymer is epoxy filled with Tungsten filler.

In yet other embodiments, the focusing of the ultrasonic wave is obtained by other methods than described above, for example using electronic focusing techniques. In this configuration, the focused acoustic source 31 is comprised from multiple piezoelectric elements that are driven by a multi-output power driver. The focusing is obtained by the introduction of a specific delay between the driving signals so that the focused wave arrives at the acoustic focal point 21 in the pre-determined manner. In some embodiments, the multi-element transducer comprises more than 1 element. In another embodiment, the transducer comprises between 2 and 256 elements.

### Other Illumination Configurations

Some other embodiments disclosed herein comprise a plurality of light sources 11 coupled to a housing 1 working in different ranges of the optical spectrum. In some embodiments, at least one light source operates in light spectrum from 450 μm to 850 μm and other light source (at least one) operates in the range from 200 μm, to 500 μm. The light sources 11 can be individually switched on and off. In some embodiments, the light sources 11 can also be operated simultaneously. In another embodiments, the light sources 11 described above are combined with another light source operating in a narrow spectrum of visible light, for example from 550 μm to 650 μm, from 450 μm to 550 μm, from 650 μm to 750 μm.

In yet other configurations, the optical monitoring system comprises at least one light polarising filter. The filter is placed along the optical axis 130 in front of the objective 115. In some embodiments, the optical separator 4 can be made so it functions also as a light polarising filter. The polarising filter is then oriented with respect to the light source so it provides a cross-polarised imaging that can be beneficial for the operator as it removes glare and reflections from the imaged surface. In some other configurations, the polarising filter and the light source are oriented in the same direction providing parallel polarisation that outline only the features of the surface. In preferred configurations, the orientation of the polarising light vs polarising filter is controllable. In some embodiments, the control is provided by a plurality of light sources distributed along the circumference of the housing. The light sources are then switched on and off to provide a light pattern that results in controllable polarization of light used in imaging.

### Handpiece

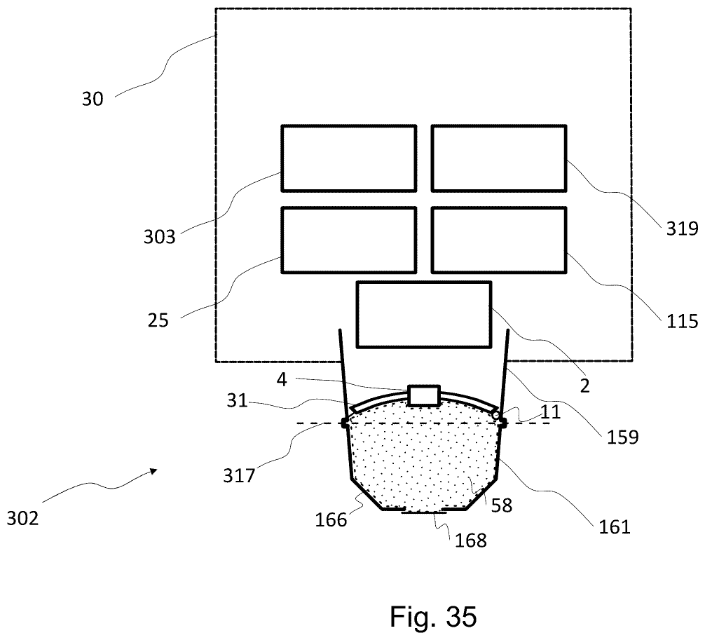

FIG. 35 illustrates elements of a handpiece. One skilled in the art can appreciate that in several embodiments, the acoustic device operates in connection with a handpiece 302, sometimes referred to as an applied part, a probe, or a wand. The handpiece is comprised of the part of a therapeutic system 300 that during the treatment are held by the medical professional administering treatment As discussed in several places herein including with respect to FIG. 4, the handpiece 302, may be comprised of a fixed piece 159 and an adjustable piece 161. In several embodiments disclosed herein, the fixed part of the housing 159 is coupled to the handpiece housing 30. In preferred embodiments, the fixed part of the housing 159 is permanently affixed to the handpiece housing 30 and the adjustable piece 161 is removably coupled to the fixed part of the housing 159.

In preferred embodiments, the following elements are contained within the fixed part of the housing 159: the focused acoustic source 31, the optical separator 4 and the electric matching circuit 2. In preferred embodiments, the handpiece controller 303, the elements of the optical observation subsystem such as the objective 115, the image sensor 25 and the auxiliary electronics 119, are either located in the fixed part of the housing 159 or are housed in the handpiece housing 30.

In many embodiments disclosed herein, the division plane 317 between the fixed part of the housing 159 and the adjustable part of the housing 161 can be located anywhere between the plane of the acousto-optic window 168 and the focused acoustic source 31. The stationary part of the housing 159 and the adjustable part of the housing 161 are coupled in such a way, so they form an impermeable chamber 166, filled with a coupling medium 58. In some embodiments, a light source 11 is coupled to the fixed part of the housing 159. In some other embodiments, it is coupled to the adjustable part of the housing 161. In yet another configuration, some light sources are coupled to the fixed part of the housing 159 and some others are coupled to the adjustable part of the housing 161.

### Therapeutic Systems

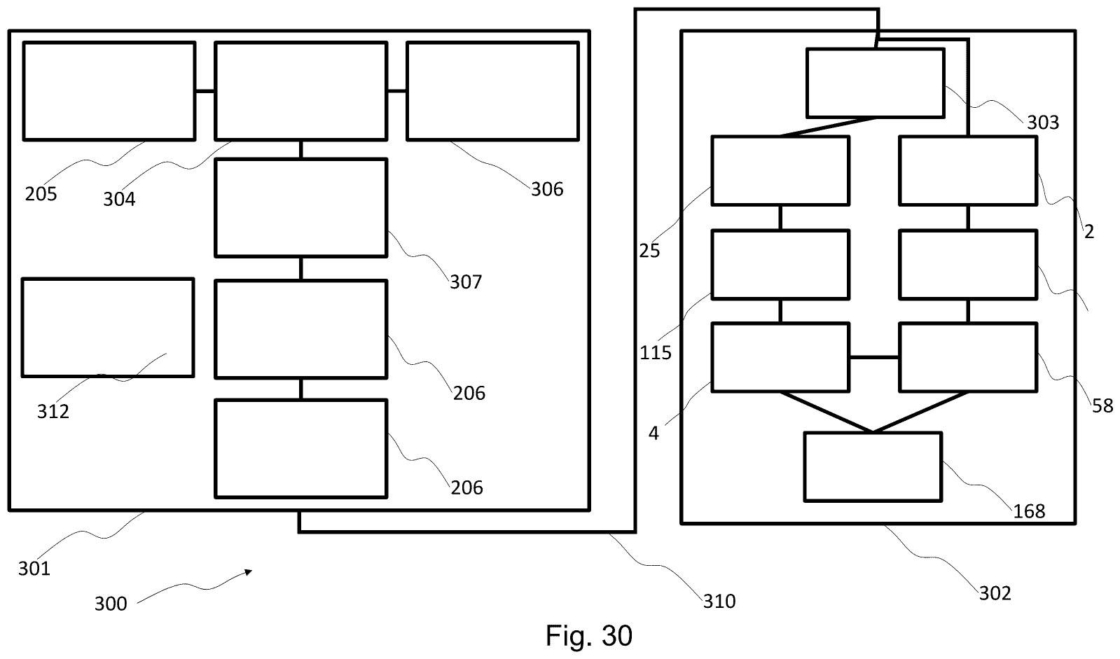

As may be seen in FIG. 30, the embodiments disclosed herein may be a part of a therapeutic system 300. In some configurations, the system 300 comprises a main unit 301 and a handpiece 302. The main unit 301 contains main controller 304 and provides subsystems for proper operation of the focused source 31, such as frequency synthesis 307, RF (Radio Frequency) power source 308 (RF power amplifier, RF power driver) and RF filtering and matching 309. In the embodiment shown in FIG. 30, the main unit 301 and the handpiece 302 are both connected by a wired connection 310. In some configurations, the wired connection has a capability of conducting RF power signals and control and digital signals as well as DC power supply for the handpiece. In preferred configuration, the wired connection provides the interface for continuous optical monitoring of the surface of the skin during the treatment presented on the display 306.

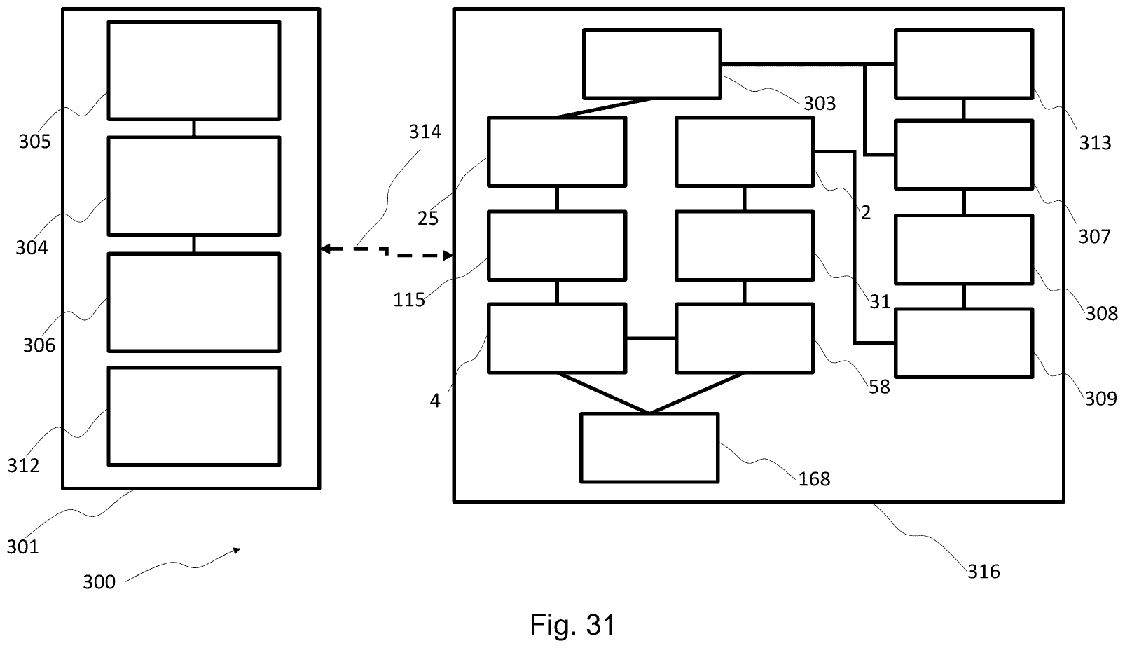

In some other embodiments disclosed herein, like the one schematically illustrated in FIG. 31, the therapeutic system 300 comprises a main unit 301 and a standalone handpiece 316. The main unit provides a user interface as well as the display functionality. The standalone handpiece is controlled through a wireless interface 314, which in the preferred configuration provides the continuous optical monitoring of the surface of the skin during the treatment presented on the display 306. The standalone handpiece is supplied internally by a battery 313.

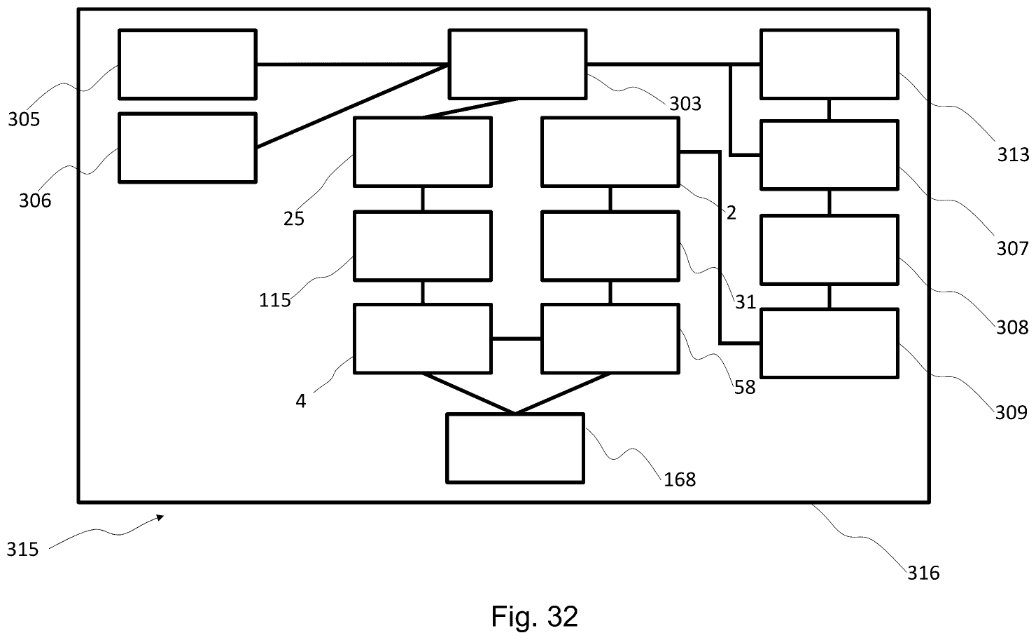

In yet another configuration, like the one schematically illustrated in FIG. 32, the embodiments disclosed herein are parts of a handheld therapeutic system 315. In configurations like the one shown in FIG. 32, all necessary subsystems (controller, frequency synthesis, RF power amplifier, RF filtering and matching) are built into the standalone handpiece 316. Moreover, the display as well as user interface functionality integrated into the standalone handpiece as well.

What is claimed is:

1. An acoustic generating system comprising:
a fixed part of a handpiece wherein the fixed part includes a focused acoustic generating source with a fixed acoustic focal length;
a plurality of adjustable parts for the handpiece wherein each adjustable part in the plurality of adjustable parts is designed to be removably and interchangeably coupled to the fixed part of the handpiece, one at a time; and
a watertight chamber formed by the fixed part and one of the plurality of adjustable parts when coupled together, wherein the watertight chamber is designed to hold a coupling medium.
wherein a penetration depth of the handpiece is adjusted by coupling different adjustable parts in the plurality of adjustable parts with the fixed part.

2. The acoustic generating system of claim 1, wherein the fixed part of the handpiece further comprises an optical system that provides imaging of a treatment surface through any adjustable part in the plurality of adjustable parts that is coupled to the fixed part.

3. The acoustic generating system of claim 2, wherein the optical system comprises an objective and an imaging plane and wherein a distance along a longitudinal axis between the objective and the imaging plane is adjustable.

4. The acoustic generating system of claim 3, wherein the distance is adjusted by mechanically moving the objective along the longitudinal axis towards or away from the imaging plane.

5. The acoustic generating system of claim 2, wherein an optical principal axis of the optical system is coaxial with a main acoustic axis of the focused acoustic generating source.

6. The acoustic generating system of claim 2, wherein the optical system comprises an imaging plane and at least one optical component with an adjustable optical focal length along a longitudinal axis.

7. The acoustic generating system of claim 1, wherein the fixed part of a handpiece inside the watertight chamber contains a bubble trap with a depth from 0.1 mm to 100 mm.

8. The acoustic generating system of claim 6, wherein the adjustable optical focal length is achieved by one of a group consisting of a liquid lens, electrowetting, use of shape-changing polymers and acousto-optical tuning.

9. The acoustic generating system of claim 2, wherein the penetration depth is adjustable in a range between at least 0 mm and 5 mm and an optical depth of field of the optical system is equal to or greater than range.

10. The acoustic generating system of claim 2, wherein the optical system has an optical main axis that includes a portion that is not coaxial with an acoustic main axis.

11. The acoustic generating system of claim 1, further comprising a fixing collar that couples an acousto-optical window to any adjustable part in the plurality of adjustable parts.

12. An acoustic generating system comprising:
a fixed part of a handpiece wherein the fixed part includes a focused acoustic generating source with a fixed acoustic focal length;
a plurality of adjustable parts for the handpiece wherein each adjustable part in the plurality of adjustable parts is designed to be removably and interchangeably coupled to the fixed part of the handpiece, one at a time and has a different length along a longitudinal axis; and
a watertight chamber formed by the fixed part and one of the plurality of adjustable parts when coupled together, wherein the watertight chamber is designed to hold a coupling medium;
an optical system affixed within the fixed part of the handpiece wherein the optical system is designed to provide imaging of a treatment surface through any adjustable part in the plurality of adjustable parts coupled to the fixed part;
wherein a penetration depth of the handpiece is adjusted by coupling different adjustable parts in the plurality of adjustable parts with the fixed part.

13. The acoustic generating system of claim 12, wherein the optical system comprises an objective and an imaging plane and wherein a distance along a longitudinal axis between the objective and the imaging plane is adjustable.

14. The acoustic generating system of claim 13, wherein the distance is adjusted by mechanically moving the objective along the longitudinal axis towards or away from the imaging plane.

15. The acoustic generating system of claim 12, wherein an optical principal axis of the optical system is coaxial with a main acoustic axis of the focused acoustic generating source.

16. The acoustic generating system of claim 12, wherein the optical system comprises an imaging plane and at least one optical component with an adjustable optical focal length along a longitudinal axis.

17. The acoustic generating system of claim 16, wherein the adjustable optical focal length is achieved by one of a group consisting of liquid lens, electrowetting, use of shape-changing polymers and acousto-optical tuning.

18. The acoustic generating system of claim 12, wherein the penetration depth is adjustable in a range between at least 0 mm and 5 mm and an optical depth of field of the optical system is equal to or greater than the range.

19. The acoustic generating system of claim 1, further comprising a fixing collar thar couples an acousto-optical window to any adjustable part in the plurality of adjustable parts.

20. An acoustic generating system comprising:
a fixed part of a handpiece; and
a plurality of adjustable parts for the handpiece wherein each adjustable part in the plurality of adjustable parts has a different length along a longitudinal axis and is designed to be removably and interchangeably coupled to the fixed part of the handpiece, one at a time.
wherein an interface between the fixed part of the handpiece and any adjustable part in the plurality of adjustable parts passes through a watertight chamber that is designed to contain a liquid acoustic coupling medium.
